(19) United States
(12) Patent Application Publication
(10) Pub. No.: US 2026/0364315 A1
(43) Pub. Date: Nov. 19, 2026

(54) CONNECTORS, CONNECTION MEDICAL WIRE, AND RELATED METHODS

(71) Applicant: Puzzle Medical Devices Inc., Montreal (CA)

(72) Inventors: Gabriel GEORGES, Sherbrooke (CA); François TRUDEAU, Québec (CA); Philippe CORMIER, Montreal (CA); Yves-Antoine CRÊTE, Montreal (CA)

(21) Appl. No.: 19/735,372

(22) Filed: Jul. 8, 2026

Related U.S. Application Data
(63) Continuation of application No. PCT/CA2025/050038, filed on Jan. 10, 2025.
(60) Provisional application No. 63/620,028, filed on Jan. 11, 2024.

(51) Int. Cl. A61M 25/09; A61M 60/865

(52) U.S. Cl. CPC A61M 25/09; A61M 60/865; A61M 2025/09175

(57) ABSTRACT

A connector for an implantable medical device includes: an electrical connector portion configured to electrically connect the implantable medical device to a controller for operating the implantable medical device through the electrical connector portion by the controller; and a wire configured to project from the electrical connector portion and the implantable medical device. Related methods of using an electrical connector of a medical device are described.

### FIELD

This disclosure relates generally to connectors, connection medical wire, and related methods.

### BACKGROUND

Advances in transcatheter technologies and techniques have enabled the implantation of an ever-increasing number of medical devices without the burden typically associated with open surgical implantations. To achieve their respective function once implanted, some of those medical devices require power, such as in the case of implantable blood pumps, whereas some other medical devices do not require power, such as in the case of prosthetic valves used in transcatheter aortic valve replacement procedures.

In cardiovascular transcatheter applications, for example, some medical device may be implanted in a subject by introducing a power wire leading end portion of the medical device through a first subject intraluminal access inside a subject vasculature, advancing the power wire along inside the subject vasculature, and externalizing the power wire leading end portion through a second subject intraluminal access. So externalized, the power wire leading end portion may be pulled through the second subject intraluminal access to moves the medical device inside the subject vasculature up to an intraluminal implantation site. Then, the power wire leading end portion may be connected to a controller for operating the medical device at the intraluminal implantation site.

However, depending on the medical procedure performed and/or the subject anatomy, the power wire of the medical device may not be long enough to run from the first subject intraluminal access to the second subject intraluminal access. As a result, the power wire leading end portion remains inside the subject vasculature and cannot be manipulated by an operator, for example by pulling on it to move the medical device intraluminally and/or by connecting it to a comptroller to operate the medical device intraluminally. In such circumstances, several medical devices, each provided with a different power wire length, would inconveniently be needed to accommodate the medical procedure performed and/or the subject anatomy.

On the other hand, if long enough to run from the first subject intraluminal access to the second subject intraluminal access, the power wire of the medical device may be of an excessive length once the medical device is implanted, as compared to the power wire length that would be normally required to operate intraluminally the medical device from outside the subject vasculature. This may be the case, for example, when the medical device is implanted closer to the second subject intraluminal access than the first subject intraluminal access. As such, the excessive length of the power wire is not practical and cumbersome for the subject.

Therefore, there is a need in the field of implantable medical devices.

### SUMMARY

An aspect on this disclosure is directed generally to a connector for an implantable medical device, including: an electrical connector portion configured to electrically connect the implantable medical device to a controller for operating the implantable medical device through the electrical connector portion by the controller; and a wire configured to project from the electrical connector portion and the implantable medical device.

The connector may include a mechanical connector portion, and the wire may include a mechanical connector configured to removably connect the mechanical connector portion.

The connector may be sized and shaped for a transcatheter application.

The wire may be configured to be routed inside a subject body.

The wire may be configured to be manipulated by an operator for moving the implantable medical device inside a subject body.

The connector may be configured to be integrated to a power wire of the implantable medical device.

Another aspect on this disclosure is directed generally to a connection medical wire, including: a medical wire; and a connector coupled to the medical wire and configured to connect to an implantable medical device.

The medical wire may be a guide wire.

The connector may be configured to connect to an electrical connector of the implantable medical device.

The connection medical wire may be configured to sustain a pulling force applied thereto that is sufficient to move the implantable medical device inside a subject body without disconnection of the connector from the implantable medical device.

The connector may be configured to be removably connectable to the implantable medical device, including the electrical connector thereof.

Another aspect on this disclosure is directed generally to a method of using an electrical connector of a medical device, the method including: manipulating a wire that projects from the electrical connector and the medical device; and electrically connecting the electrical connector to a controller configured to electrically operate the medical device through the electrical connector.

Manipulating may include disconnecting the wire from the electrical connector.

Manipulating may include cutting the wire to remove at least a portion thereof from the electrical connector.

Manipulating may include folding the wire.

Manipulating may include passing the wire through an electrical connector of the controller.

The method of using an electrical connector of a medical device may include: transcatheterly implanting the medical device by introducing the wire through a first subject intraluminal access; routing the wire inside a subject body up to a second subject intraluminal access; externalizing the wire through the second subject intraluminal access; and pulling the wire through the second subject intraluminal access to externalize the electrical connector.

The method of using an electrical connector of a medical device may include: electrically operating the medical device inside a subject body by the controller located outside the subject body.

Another aspect on this disclosure is directed generally to a method of using an electrical connector of a medical device implanted inside a subject body, the method including: connecting a wire to the medical device; and explanting the medical device from the subject body to leave the wire routed inside the subject body.

Connecting includes connecting the wire to a portion of the medical device that is externalized through a subject intraluminal access.

The portion of the medical device may include an electrical connector.

According to the method of using an electrical connector of a medical device implanted inside a subject body, the medical device may be operated inside the subject body through the electrical connector by a controller electrically connected to the electrical connector. The method may include: electrically disconnecting the electrical connector of the medical device and the controller from each other before connecting the wire to the medical device.

The method of using an electrical connector of a medical device implanted inside a subject body may include: stopping the operation of the medical device.

Explanting may include explanting the medical device through another subject intraluminal access.

According to the method of using an electrical connector of a medical device implanted inside a subject body, the wire may be routed inside the subject body between the subject intraluminal access and the other subject intraluminal access after transcatheter explantation of the medical device.

Another aspect on this disclosure is directed generally to a connector for an implantable medical device electrically operable by a power wire thereof, the connector including: an electrical connector portion configured to couple the power wire and to electrically connect the implantable medical device to a controller for electrically operating the implantable medical device by the controller; and a mechanical connector portion configured to mechanically connect the implantable medical device to a connection medical wire, the mechanical connector portion being capable of sustaining a pulling force applied to the connection medical wire for moving the implantable medical device inside a lumen of a subject body conduit when the mechanical connector portion is mechanically connected to the connection medical wire.

The connector may include a controller-connecting end portion that is associated with the mechanical connector portion.

The connector may include a tensile wire configured to couple the connector and the implantable medical device.

The tensile wire may be coupled to the mechanical connector portion.

The tensile wire may be coupled to the electrical connector portion.

The mechanical connector portion may be capable of sustaining the pulling force without disconnecting from the connection medical wire when mechanically connected thereto.

The connector may be capable of sustaining the pulling force without breaking apart when the mechanical connector portion is mechanically connected to connection medical wire.

The connector may be sized to be received inside the lumen of the subject body conduit.

Another aspect on this disclosure is directed generally to a power wire that may include: a connector as described herein.

Another aspect on this disclosure is directed generally to an implantable electrically operated blood pump that may include a connector as described herein.

Another aspect on this disclosure is directed generally to a connection medical, the connection medical may include: a mechanical connector configured to mechanically connect to an implantable medical device; and a medical wire projecting from the mechanical connector, the medical wire being configured to be navigated inside a lumen of a subject body conduit and also to be manipulated for moving the implantable medical device inside the lumen of the subject body conduit when the mechanical connector is connected to the implantable medical device.

The connection medical wire may be capable of sustaining a pulling force applied to the medical wire for moving the implantable medical device inside the lumen of the subject body conduit when the mechanical connector is connected to the implantable medical device.

The mechanical connector may be capable of sustaining the pulling force without disconnecting from the implantable medical device when connected thereto.

The medical wire may be capable of sustaining the pulling force without breaking apart when the mechanical connector is connected to the implantable medical device.

The mechanical connector may be sized to be received inside the lumen of the subject body conduit.

Another aspect on this disclosure is directed generally to an electrical connector for an implantable medical device electrically operable by a power wire thereof, the electrical connector may include: a body configured to couple the power wire and to electrically connect the implantable medical device to a controller for electrically operating the implantable medical device by the controller; and a connector wire projecting from the body and configured to be navigated inside a lumen of a subject body conduit and also to be manipulated for moving the implantable medical device inside the lumen of the subject body conduit when the electrical connector is coupled to the implantable medical device.

The body may include a controller-connecting end portion that is associated with the connector wire.

The electrical connector may be capable of sustaining a pulling force applied to the connector wire for moving the implantable medical device inside the lumen of the subject body conduit when the electrical connector is coupled to the implantable medical device.

The electrical connector may include a tensile wire coupled to the body and projecting in a direction opposite to the connector wire.

The tensile wire may project in a same direction as the power wire when the power wire is coupled to the body.

The electrical connector may be capable of sustaining the pulling force without breaking apart when the electrical connector is coupled to the implantable medical device.

The connector wire may be rotatable relative to the body.

The connector wire may be coupled to the body, extends along the body, and projects outwardly from the body in opposite directions thereof.

A portion of the connector wire may project in a same direction as the power wire when the power wire is coupled to the body.

The electrical connector may be capable of sustaining the pulling force without breaking apart when the electrical connector is coupled to the implantable medical device.

Another aspect on this disclosure is directed generally to a power wire that may include the electrical connector as described herein.

Another aspect on this disclosure is directed generally to a an implantable electrically operated blood pump that may include the electrical connector as described herein.

Another aspect on this disclosure is directed generally to a method of using a medical device inside a lumen of a subject body conduit, the method may include: navigating a wire from a first subject intraluminal access to a second subject intraluminal access inside a lumen of a subject body conduit; and manipulating the wire, which is coupled to a medical device, from the second subject intraluminal access to move the medical device inside the lumen of the subject body conduit.

Navigating the wire may include: introducing a leading end portion of the wire through the first subject intraluminal access and advancing the leading end portion of the wire along the lumen of the subject body conduit toward the second subject intraluminal access.

Navigating may include: externalizing the leading end portion of the wire from the second subject intraluminal access.

Manipulating may include: manipulating a wire portion outwardly projecting from inside the lumen of the subject body conduit.

Manipulating may include: pulling the wire through the second subject intraluminal access to cause at least one of (i) introducing the medical device through the first subject intraluminal access inside the lumen of the subject body conduit, and (ii) moving the medical device inside the lumen of the subject body conduit.

Connecting may include: connecting the wire to a controller-connecting end portion of the medical device.

Connecting may include: connecting the wire to a power wire of the medical device that is used to electrically operate the medical device.

Connecting may include: connecting the wire to an electrical connector of the medical device.

Connecting may include: mechanically connecting the wire to the medical device only when the medical device is in an electrically unconnected state.

The method of using a medical device inside a lumen of a subject body conduit may include: operatively connecting the medical device to a controller to operate the medical device inside the lumen of the subject body conduit by the controller.

Operatively connecting the medical device to the controller may include: electrically connecting the medical device to a controller to operate the medical device inside the lumen of the subject body conduit by the controller.

Features and advantages of the disclosed subject-matter will become apparent in view of the following detailed description of selected embodiments, as illustrated in the accompanying drawings.

### BRIEF DESCRIPTION OF THE DRAWINGS

In order that this disclosure be readily understood, at least some selected embodiments thereof are illustrated by way of example(s) in the accompanying drawings. Accordingly, the accompanying drawings are illustrative in nature only and are not intended to be construed and interpreted as limiting the extent of the subject matter protected by the claims.

It is noted that like reference characters identify similar or equivalent feature(s) throughout the drawings. If present in the claims, reference character(s) is/are provided only to make claims easier to comprehend and are not intended to be construed and interpreted as limiting the extent of the subject matter protected by the claims. Also, the features illustrated throughout the drawings are not necessarily drawn to scale.

To help understand the drawings and figures, element(s) and/or feature(s) that may be optional to the technology disclosed may be represented in dashed lines.

FIGS. 1-3 are schematic representations of a bifunctional connector and a connection medical wire, in accordance with a first and a second aspects of the disclosed technology, respectively. FIG. 1 schematically represents the bifunctional connector 100 and the connection medical wire 108 in an unconnected state with the bifunctional connector 100 and the connection medical wire 108 unconnected from each other, in accordance with one or more embodiment(s). FIG. 2 schematically represents the bifunctional connector 100 and the connection medical wire 108 in a connected state with the bifunctional connector and the connection medical wire mechanically connected together, in accordance with one or more embodiment(s). FIG. 3 schematically represents the bifunctional connector and an optional controller in a connected state with the bifunctional connector and the optional controller electrically connected together, in accordance with one or more embodiment(s).

FIGS. 4-6 are perspective and longitudinal cross-sectional views of the bifunctional connector, such as one of FIGS. 1-3, provided with a ball connector, and of the connection medical wire, such as one of FIGS. 1-3, provided with a socket connector, in accordance with one or more embodiment(s).

FIGS. 7-8 are perspective and longitudinal cross-sectional views of the bifunctional connector, such as one of FIGS. 1-3, provided with a latch connector, and of the connection medical wire, such as one of FIGS. 1-3, provided with a catch connector, in accordance with one or more embodiment(s).

FIGS. 9-10 are perspective views of the bifunctional connector, such as one of FIGS. 1-3, provided with a loop connector, and of the connection medical wire, such as one of FIGS. 1-3, provided with a hook connector, in accordance with one or more embodiment(s).

FIG. 11 is a perspective view of the bifunctional connector, such as one of FIGS. 1-3, provided with a loop connector, and of a connection catheter, in accordance with one or more embodiment(s).

FIGS. 12-13 are perspective and longitudinal cross-sectional views of the bifunctional connector, such as one of FIGS. 1-3, provided with a threaded connector, and of the connection medical wire, such as one of FIGS. 1-3, provided with a correspondent threaded connector, in accordance with one or more embodiment(s).

FIGS. 14-15 are schematic representations of an electrical connector, in accordance with a third aspect of the disclosed technology. FIG. 14 schematically represents the electrical connector in an unconnected state and having a connector wire attached thereto, in accordance with one or more embodiment(s). FIG. 15 schematically represents an electrical connector in a connected state and having the connector wire removed therefrom, in accordance with one or more embodiment(s). The electrical connector is connected to a controller for operating an optional implantable medical device. Also schematically represented are an optional tensile wire and an optional power wire, in accordance with one or more embodiment(s).

FIGS. 16-18 are perspective and longitudinal cross-sectional views of an electrical connector, such as one of FIGS. 14-15, in accordance with one or more embodiment(s).

FIGS. 19-20 are schematic representations of a cardiovascular transcatheter application using a bifunctional connector and a connection medical wire, such as the ones of FIGS. 1-3, or an electrical connector, such as one of FIGS. 14-15. in accordance with one or more embodiment(s).

FIG. 21 is a schematic representation of a method of using an electrical connector, such as the one of FIGS. 1-3 and the one of FIGS. 14-15, of a medical device, in accordance with a fourth aspect of the disclosed technology.

FIG. 22 is a schematic representation of a method of using an electrical connector, such as the one of FIGS. 1-3, of a medical device implanted inside a subject body, in accordance with a fifth aspect of the disclosed technology.

### DETAILED DESCRIPTION

The subject matter of this disclosure is described and explained in the following detailed description with reference to the non-limiting aspect(s), embodiment(s), example(s), feature(s), element(s), and step(s), as the case may be, presented hereinafter and illustrated in the accompanying non-limiting drawings and/or figures. Recognizing that these may vary, the skilled addressee shall readily understand that any other variants thereof and any combination of these other variants, as the case may be, are contemplated without departing from the scope of this disclosure, even if they are all not explicitly presented and stated herein.

Therefore, these non-limiting aspect(s), embodiment(s), example(s), feature(s), and/or element(s) is/are intended merely to facilitate an understanding of ways in which the claimed subject matter may be reduced to practice by the skilled addressee. Accordingly, these shall not to be construed as limiting the scope of the claimed subject matter, which is defined solely by the accompanying claims and applicable law.

The terminology used herein is only for the purpose of describing and explaining the claimed subject matter and is not intended to limit the scope hereof. Unless defined otherwise, all technical, engineering, scientific, and other relevant terminology used herein have the same meanings as commonly understood by the skilled addressee.

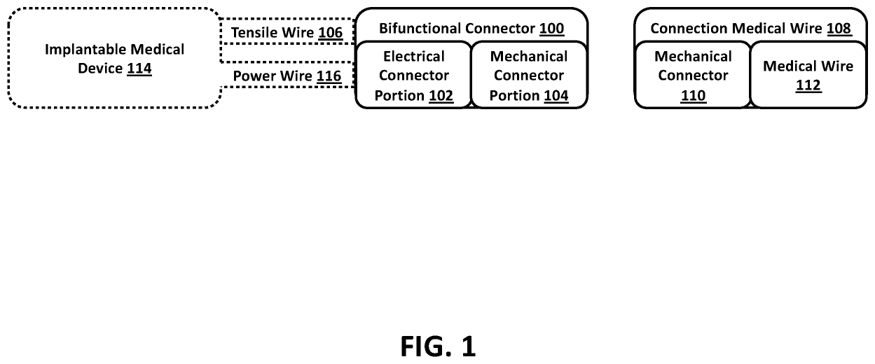

With the foregoing in mind, referring to FIGS. 1-3, this disclosure relates generally to a bifunctional connector 100 for an optional implantable medical device 114, and a connection medical wire 108 (also referred to herein as a “wire 108”) for connection to the bifunctional connector 100, according to a first and second aspects of the disclosed technology, respectively. When connected to the bifunctional connector 100 provided to an optional power wire 106, the connection medical wire 108 may generally increase the useful length and reach of the optional power wire 116 for in vivo application, as described hereinafter. For example, the connection medical wire 108 may be manipulable by an operator to move the optional implantable medical device 114 inside a lumen of a subject body conduit. The bifunctional connector 100 may also establish an electrical connection between the optional implantable medical device 114 and an optional controller 300 for operating the optional implantable medical device 114 by the optional controller 300 through the bifunctional connector 100.

In particular, FIG. 1 schematically represents the bifunctional connector 100 in a mechanically and electrically unconnected state, according to one or more embodiment(s). As such, the bifunctional connector 100 and the connection medical wire 108 are mechanically unconnected from each other, and the bifunctional connector 100 is electrically unconnected from a controller (not shown).

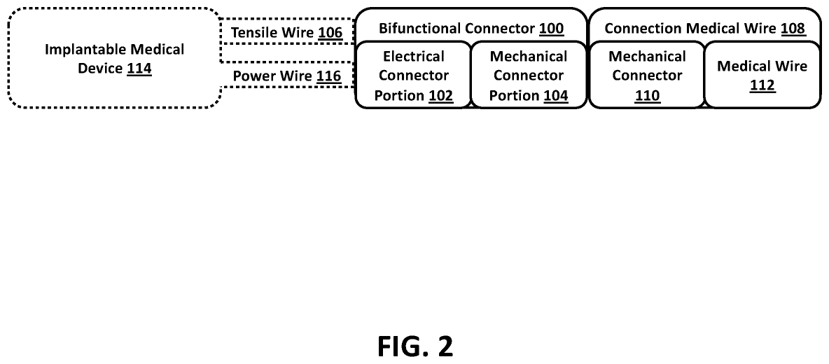

FIG. 2 schematically represents the bifunctional connector 100 in a mechanically connected state and in an electrically unconnected state, according to one or more embodiment(s). In the mechanically connected state, the mechanical connector portion 104 of the bifunctional connector 100 and the mechanical connector 110 (also referred to herein as a “connector”) of the connection medical wire 108 are connected together, such as mechanically connected together. In the electrically unconnected state, the bifunctional connector 100 is electrically unconnected from a controller (not shown). The mechanical connector portion 104 and the mechanical connector 110 may be removably connectable together.

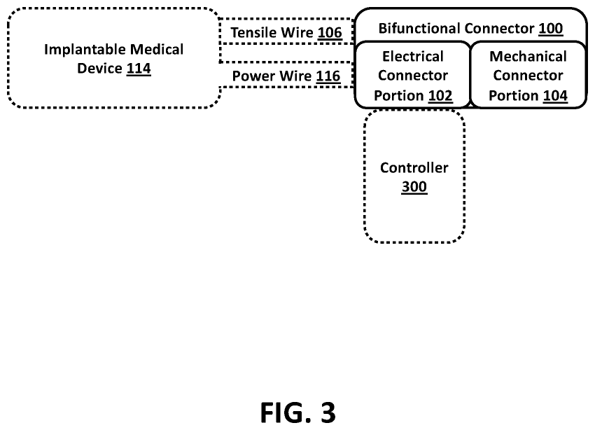

FIG. 3 schematically represents the bifunctional connector 100 in a mechanically unconnected state and in an electrically connected state, according to one or more embodiment(s). In the mechanically unconnected state, the bifunctional connector 100 and the connection medical wire 108 are mechanically unconnected from each other. In the electrically unconnected state, the electrical connector portion 102 of the bifunctional connector 100 is electrically connected to an optional controller 300 for electrically operating the optional implantable medical device 114 through the bifunctional connector 100 by the controller 300.

Throughout FIGS. 1-3, the optional tensile wire 106 and the optional power wire 116 are represented as separate structures, but they may be integrated together as illustrated in one or more of the embodiment(s) described hereinafter.

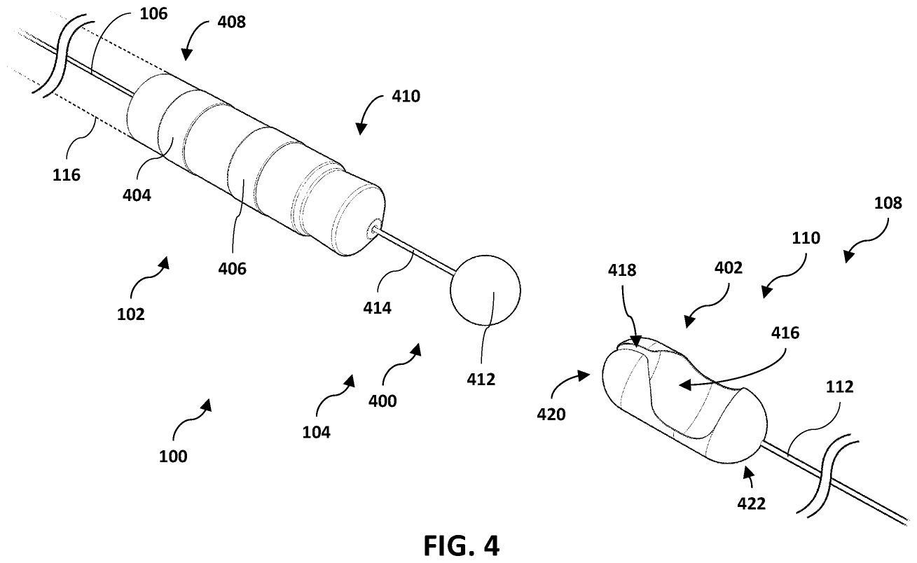

FIGS. 4-6 illustrate the bifunctional connector 100 having the mechanical connector portion 104 provided as a ball connector 400, and the connection medical wire 108 having the mechanical connector 110 provided as a socket connector 402 configured to mechanically engage and connect to the ball connector 400, according to one or more embodiment(s).

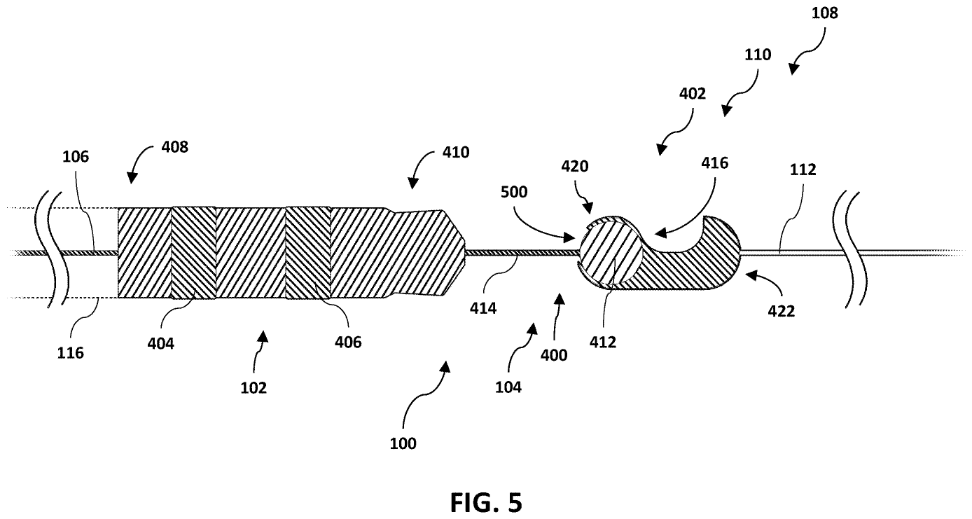

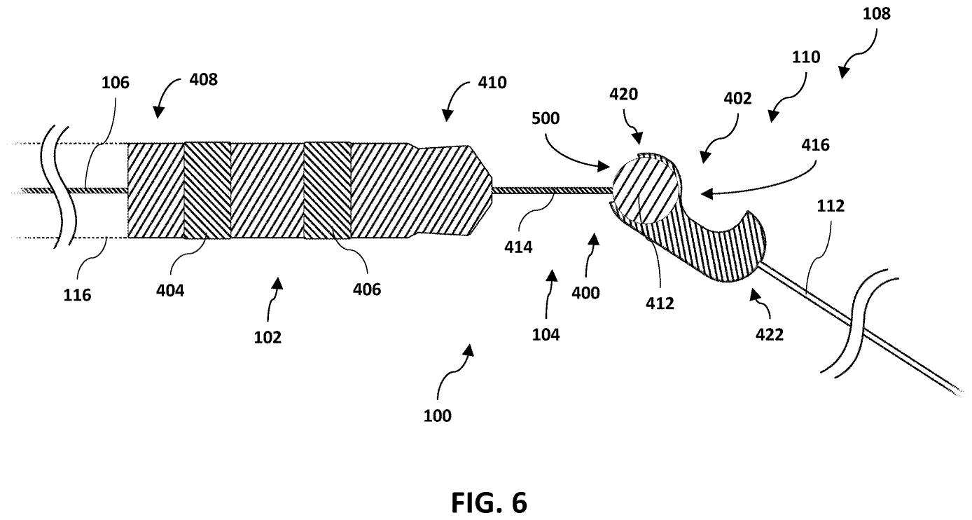

Also illustrated in FIGS. 4-6 is the electrical connector portion 102 having a cylindrical shape and two peripheral electrical pins 404, 406 disposed between a power wire-coupling end portion 408 to which the power wire 116 is coupled and a controller-connecting end portion 410 to which the ball connector 400 is associated. The power wire 116 may be coupled, either directly or indirectly, to the power wire-coupling end portion 408 notably by being welded, screwed, glued, and/or crimped thereto. FIG. 4 illustrates the ball connector 400 and the socket connector 402 not connected to each other and thus in an unconnected state. FIGS. 5-6 illustrate the ball connector 400 and the socket connector 402 connected to each other and thus in a connected state. In FIGS. 4-6, the electrical leads are omitted from the power wire 116 for the sake of clarity.

As illustrated in FIGS. 4-6, the ball connector 400 includes a ball member 412 and a pole member 414 linking the ball member 412 to the controller-connecting end portion 410 of the bifunctional connector 100. The ball connector 400 may be attached to the controller-connecting end portion 410, such as by being welded, screwed, glued, and/or crimped thereto. Alternatively, the ball connector 400 and the controller-connecting end portion 410 may form an integral, one-piece construction. As illustrated, the pole member 414 projects coaxially outwardly from the controller-connecting end portion 410. Alternatively, the pole member 414 may project non-coaxially outwardly and/or may project at an angle from the controller-connecting end portion 410. In any case, the pole member 414 has an appropriate length and positions the ball member 412 for mechanical engagement and connection to the socket connector 402, as described hereinafter.

As also illustrated, the socket connector 402 of the connection medical wire 108 has a cylindrical shape and defines a cavity 416 and slot 418 extending at least partially along the socket connector 402 between the cavity 416 and a free end portion 420 of the socket connector 402. The medical wire 112 of the connection medical wire 108 projects from a medical wire-attachment end portion 422 of the socket connector 402 and may be rotatably or non-rotatably attached thereto. The medical wire 112 may be rotatably attached by a captive attachment that rotatably retains an end portion of the medical wire 112 in the socket connector 402. Such rotatable attachment is advantageous, as compared to a non-rotatable attachment, for preventing the medical wire 112 from tangling when being rotated, as it may occur when the bifunctional connector 100 is manipulated inside a lumen of a subject body conduit. Each of the connection medical wire 108 and the medical wire 112 may be configured to be cut for removing at least a portion thereof from the bifunctional connector 100 without the need of disconnecting the mechanical connector portion 104 of the bifunctional connector 100 and the mechanical connector 110 of the connection medical wire 108 from each other.

The ball connector 400 and the socket connector 402 are connected together by inserting the ball connector 400 inside the cavity 416 and by sliding the pole member 414 along the slot 418 until appropriate mechanical engagement is achieved. When connected to the socket connector 402 and the connection medical wire 108 is pulled relative to the socket connector 402, the ball member 412 abuts toward the free end portion 420 of the socket connector 402 inside the cavity 416, as illustrated in FIG. 5. The ball connector 400 may be pushed relative to the socket connector 402 such that the ball member 412 is displaced inside the cavity 416 by a distance generally corresponding to the length of the pole member 414.

Received in the cavity 416, the ball member 412 may also rotate relative to the socket connector 402, which rotatable connection enables the advantage(s) described hereinbefore. An easier, smoother rotation of the mechanical connector portion 104 of the bifunctional connector 100 relative to the mechanical connector 110 of the connection medical wire 108 may be advantageously obtained by implementing both the captive attachment, as described hereinbefore, and a connector member capable of rotation inside the cavity 416, such as the ball member 412, as compared to implementing only one of these feature.

The ball connector 400 and the socket connector 402 are disconnected from each other by sliding the pole member 414 along the slot 418 until mechanical disengagement and removing the ball member 412 from the cavity 416.

As illustrated in FIGS. 5-6, the slot 418 may define an aperture 500 at the free end portion 420 of the socket connector 402. The aperture 500 has the clearance required for the pole member 414 to pivot relative to the socket connector 402, providing an articulated connection, as illustrated in FIG. 6 where the socket connector 402 is at an angle relative to the mechanical connector portion 104. Such articulated connection is advantageous when the ball connector 400 connected to the socket connector 402 need to be passed along a tortuous lumen of a subject body conduit, for example when the subject body conduit has an acute angle. In this circumstance, an unarticulated connection may not be able to pass the acute angle, while an articulated connection may.

The ball member 412 and the cavity 416 may be sized and shaped for an interference fit engagement therebetween. Additionally or alternatively, the pole member 414 and slot 418 may be sized and shaped for an interference fit engagement therebetween.

Still additionally or alternatively, a magnet (not shown) may be provided to one or more of the ball connector 400 and the socket connector 402 for maintaining or assisting to maintain an appropriate mechanical engagement therebetween. For example, the magnet may be configured to magnetically bias the ball connector 400 toward the cavity 416.

Although the mechanical connector portion 104 of the bifunctional connector 100 is illustrated as the ball connector 400 and the mechanical connector 110 of the connection medical wire 108 is illustrated as the socket connector 402, the mechanical connector portion 104 of the bifunctional connector 100 may be provided as the socket connector 402 and the mechanical connector 110 of the connection medical wire 108 may be provided as the ball connector 400, with the necessary change(s), appreciable to the skilled addressee, having been made, if applicable.

Also, the ball member 412 may be of any shape, such as an oblong shape and the like, that allows mechanical engagement and connection to the socket connector 402, which in this case has a complementary geometrical shape, without departing from the scope of this disclosure. It will be appreciated that some geometrical sizes and/or shapes of the ball member 412, such as a square shape, may not allow the rotation of the ball member 412 inside the cavity 416 of the socket connector 402.

Still referring to FIGS. 4-6, the power wire-coupling end portion 408 of the bifunctional connector 100 is configured to couple the power wire 116 of the implantable medical device 114 (not shown), such as by being welded, screwed, glued, and/or crimped thereto, according to one or more embodiment(s). When so coupled, each one of the two peripheral electrical pins 404, 406 of the electrical connector portion 102 is in electrical communication with a respective electrical lead (not shown) of the power wire 116, such that the implantable medical device 114 may be electrically operated by the optional controller 300 through the electrical connector portion 102. For example, the implantable medical device may be operated inside a lumen of a subject body conduit while the optional controller 300 may be located outside a subject body.

As illustrated, the peripheral electrical pins 404, 406 run peripherally at least partially about the outer periphery of the electrical connector portion 102. The peripheral electrical pins 404, 406 may be spring-loaded and outwardly biased relative to the electrical connector portion 102. Two peripheral electrical pins 404, 406 are illustrated; however, any number of peripheral electrical pins may be provided to the electrical connector portion 102.

A magnet (not shown) may be provided to electrical connector portion 102 for maintaining or assisting to maintain an appropriate electrical engagement with a connector of the optional controller 300 (also referred to herein as a “controller electrical connector”). For example, the magnet may be configured to magnetically bias the electrical connector portion 102 toward the controller electrical connector.

The electrical connector portion 102 is electrically connectable to a controller electrical connector by inserting and pushing the electrical connector portion 102 into the controller electrical connector until an appropriate electrical connection is established for operation.

The electrical connector portion 102 is disconnectable from the controller electrical connector by pulling on the electrical connector portion 102 to interrupt electrical communication with the controller electrical connector.

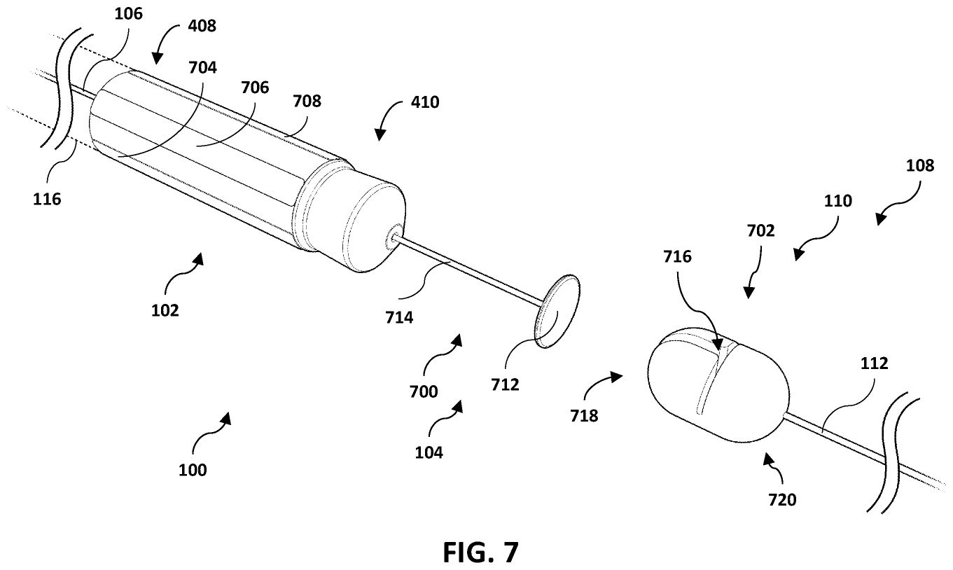

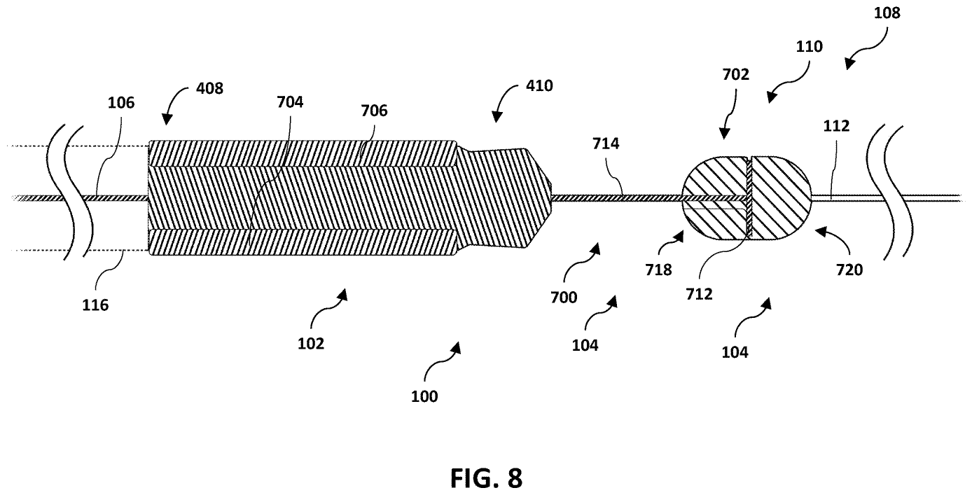

FIGS. 7-8 illustrate the bifunctional connector 100 having the mechanical connector portion 104 provided as a latch connector 700 and the connection medical wire 108 having the mechanical connector 110 provided as a catch connector 702 configured to mechanically engage and connect to the latch connector 700, according to one or more embodiment(s).

Also illustrated in FIGS. 7-8 is the electrical connector portion 102 of a cylindrical shape and having four longitudinal electrical pins 704, 706, 708, 710 disposed between the power wire-coupling end portion 408 to which the power wire 116 is coupled and the controller-connecting end portion 410 to which the latch connector 700 is associated (only longitudinal electrical pins 704, 706, 708 are visible on FIG. 7, and only longitudinal electrical pins 704, 706 are visible on FIG. 8). The power wire 116 may be coupled, either directly or indirectly, to the power wire-coupling end portion 408 notably by being welded, screwed, glued, and/or crimped thereto. FIG. 7 illustrates the latch connector 700 and the catch connector 702 not connected to each other and thus in an unconnected state. FIG. 8 illustrate the latch connector 700 and the catch connector 702 connected to each other and thus in a connected state. In FIGS. 7-8, the electrical leads are omitted from the power wire 116 for the sake of clarity.

As illustrated in FIGS. 7-8, the latch connector 700 includes a disc member 712 and a pole member 714 linking the disc member 712 to the controller-connecting end portion 410 of the bifunctional connector 100. The latch connector 700 may be attached to the controller-connecting end portion 410, such as by being welded, screwed, glued, and/or crimped thereto. Alternatively, the latch connector 700 and the controller-connecting end portion 410 may form an integral, one-piece construction. As illustrated, the pole member 714 projects coaxially outwardly from the controller-connecting end portion 410. Alternatively, the pole member 714 may project non-coaxially outwardly and/or may project at an angle from the controller-connecting end portion 410. In any case, the pole member 414 has an appropriate length and positions the disc member 712 for mechanical engagement and connection to the catch connector 702, as described hereinafter.

As also illustrated, the catch connector 702 of the connection medical wire 108 has a cylindrical shape and defines a T-shaped groove 716 extending at least partially along the catch connector 702 between a free end portion 718 and a medical wire-attachment end portion 720 thereof. The medical wire 112 of the connection medical wire 108 projects from a medical wire-attachment end portion 720 of the catch connector 702 and may be rotatably attached, such as by a captive attachment as described hereinbefore, or non-rotatably attached thereto. The rotatable attachment enabling the advantage(s) described hereinbefore.

The latch connector 700 and the catch connector 702 are connected together by inserting the disc member 712 and the pole member 714 inside the T-shaped groove 716 until appropriate mechanical engagement is achieved. When connected to the catch connector 702 and the connection medical wire 108 is pulled relative to the catch connector 702, the disc member 712 abuts toward the free end portion 718 of the catch connector 702 inside the T-shaped groove 716.

Received in the T-shaped groove 716, the disc member 712 and/or pole member 714 may also rotate relative to the catch connector 702, enabling the advantage(s) described hereinbefore, including when both the captive attachment and a connector member capable of rotation inside the T-shaped groove 716, such as the disc member 712, are implemented.

The latch connector 700 and the catch connector 702 are disconnected from each other by removing or mechanically disengaging the disc member 712 and the pole member 714 from the T-shaped groove 716.

The disc member 712, the pole member 714, and the T-shaped groove 716 may be sized and shaped for an interference fit engagement therebetween. Additionally or alternatively, a magnet (not shown) may be provided to one or more of the disc member 712, the pole member 714, and the catch connector 702 for maintaining or assisting to maintain an appropriate mechanical engagement therebetween. For example, the magnet may be configured to magnetically bias one or both of the disc member 712 and the pole member 714 toward the T-shaped groove 716.

Also, the disc member 712 may be of any shape, such as an oblong shape and the like, that allows mechanical engagement and connection to the catch connector 702, which in this case has a complementary geometrical shape, without departing from the scope of this disclosure. It will be appreciated that some geometrical sizes and/or shapes of the disc member 712, such as a square shape, may not allow the rotation of the disc member 712 inside the T-shaped groove 716 of the catch connector 702.

Still referring to FIGS. 7-8, the longitudinal electrical pins 704-710 run longitudinally at least partially along the length of the electrical connector portion 102. The longitudinal electrical pins 704-710 may be spring-loaded and outwardly biased relative to the electrical connector portion 102. Four longitudinal electrical pins 704-710 may be provided to the electrical connector portion 102; however, any number of longitudinal electrical pins may be provided to the electrical connector portion 102.

The electrical connector portion 102 provided with the longitudinal electrical pins 704-710 being generally similar to the electrical connector portion 102 provided with the peripheral electrical pins 404, 406, the electrical connector portion 102 provided with the longitudinal electrical pins 704-710 will not be further described herein for the sake of brevity. It will be appreciated that a similar description applies between the electrical connector portion 102 provided with the longitudinal electrical pins 704-710 and the electrical connector portion 102 provided with the peripheral electrical pins 404, 406, with the necessary change(s), appreciable to the skilled addressee, having been made, if applicable.

The electrical connector portion 102 provided with longitudinal electrical pins may be further provided with an engagement means configured to engage with a corresponding connector, such as a controller electrical connector, in a way that each longitudinal electrical pin of the electrical connector portion 102 can only contact a respective one of the electrical pins of the corresponding connector without contacting the other ones to ensure electrical contact specificity. The engagement means may include any cross-sectional shape of the bifunctional connector 100, including the electrical connector portion 102 and/or the mechanical connector portion 104, that is complementary to a cross-sectional shape of the corresponding connector. The engagement means may also include a depression, such as a groove, a notch, and the like, and/or a protuberance, such as a bulge, a knob, and the like.

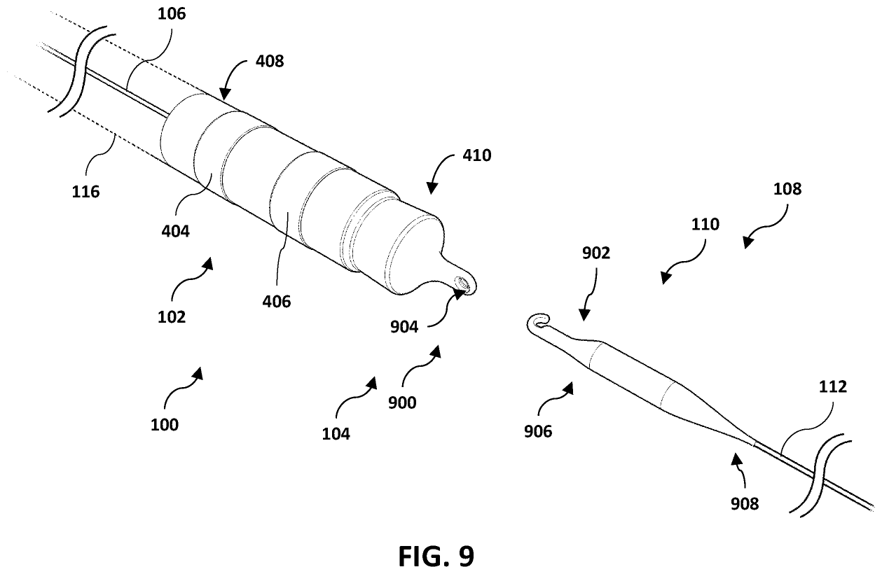

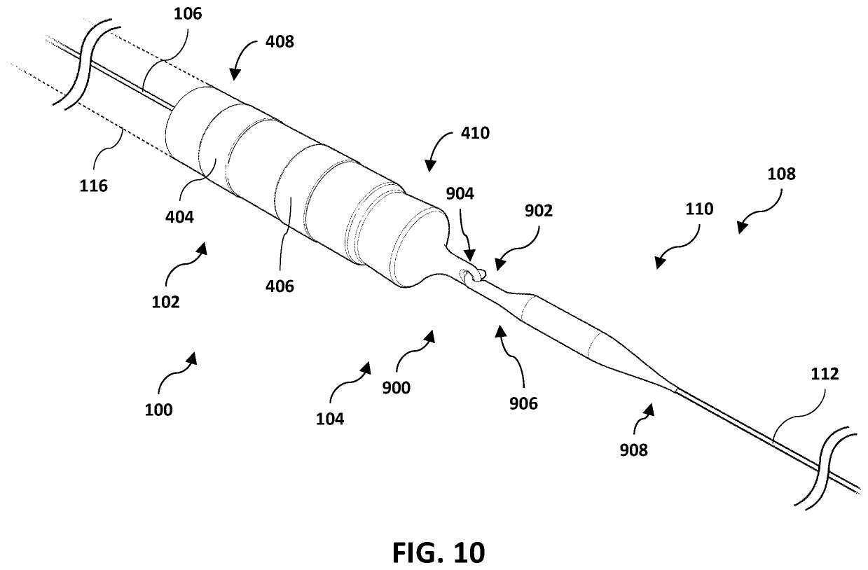

FIGS. 9-10 illustrate the bifunctional connector 100 having the mechanical connector portion 104 provided as a loop connector 900, and the connection medical wire 108 having the mechanical connector 110 provided as a hook connector 902 configured to mechanically engage and connect to the loop connector 900, according to one or more embodiment(s).

Also illustrated in FIGS. 9-10, similarly to FIGS. 4-6, is the electrical connector portion 102 having a cylindrical shape and two peripheral electrical pins 404, 406 disposed between a power wire-coupling end portion 408 to which the power wire 116 is coupled and a controller-connecting end portion 410 to which the loop connector 900 is associated. The power wire 116 may be coupled, either directly or indirectly, to the power wire-coupling end portion 408 notably by being welded, screwed, glued, and/or crimped thereto. FIG. 9 illustrates the loop connector 900 and the hook connector 902 not connected to each other and thus in an unconnected state. FIG. 10 illustrate the loop connector 900 and the hook connector 902 connected to each other and thus in a connected state.

As illustrated in FIGS. 9-10, the loop connector 900 projects coaxially outwardly from the controller-connecting end portion 410. Alternatively, the loop connector 900 may project non-coaxially outwardly and/or may project at an angle from the controller-connecting end portion 410. The loop connector 900 may be attached to the controller-connecting end portion 410, such as by being welded, screwed, glued, and/or crimped thereto. Alternatively, the loop connector 900 and the controller-connecting end portion 410 may form an integral, one-piece construction. In any case, the loop connector 900 has an appropriate length and positions a hole 904 thereof for mechanical engagement and connection to the hook connector 902.

As also illustrated in FIG. 10, the hook connector 902 and the hole 904 of the loop connector 900 are configured such that a portion of the hook connector 902 is receivable into the hole 904 for connection. The hook connector 902 may include a gate member (not shown) for preventing unwanted disconnection of the loop connector 900 from the hook connector 902 when the hook connector 902 is received into the hole 904. The gate member has an open position for the hook connector 902 to be received into the hole 904, and a close position for preventing unwanted disconnection therebetween. The gate member may be spring-loaded toward a close position for preventing unwanted disconnection. The medical wire 112 of the connection medical wire 108 projects from a medical wire-attachment end portion 908 of the hook connector 902 and may be rotatably attached, such as by a captive attachment as described hereinbefore, or non-rotatably attached thereto. The rotatable attachment enabling the advantage(s) described hereinbefore.

The hook connector 902 and the loop connector 900 are connected together by inserting a portion of the hook connector 902 into the hole 904 of the loop connector 900 until appropriate mechanical engagement is achieved.

The hook connector 902 and the loop connector 900 are disconnected from each other by removing a portion of the hook connector 902 from the hole 904 of the loop connector 900 until mechanical disengagement.

When present, the gate member is actuatable toward the open position for inserting a portion of the hook connector 902 into the hole 904 and for removing a portion of the hook connector 902 from the hole 904.

The hook connector 902 and the hole 904 of the loop connector 900 may be sized and shaped for an interference fit engagement therebetween.

Additionally or alternatively, a magnet (not shown) may be provided to one or more of the hook connector 902 and the socket connector 402 for maintaining or assisting to maintain an appropriate mechanical engagement therebetween. For example, the magnet may be configured to magnetically bias the hook connector 902 toward the hole 904 of the loop connector 900.

Although the mechanical connector portion 104 of the bifunctional connector 100 is illustrated as the loop connector 900 and the mechanical connector 110 of the connection medical wire 108 is illustrated as the hook connector 902, the mechanical connector portion 104 of the bifunctional connector 100 may be provided as the hook connector 902 and the mechanical connector 110 of the connection medical wire 108 may be provided as the loop connector 900, with the necessary change(s), appreciable to the skilled addressee, having been made, if applicable.

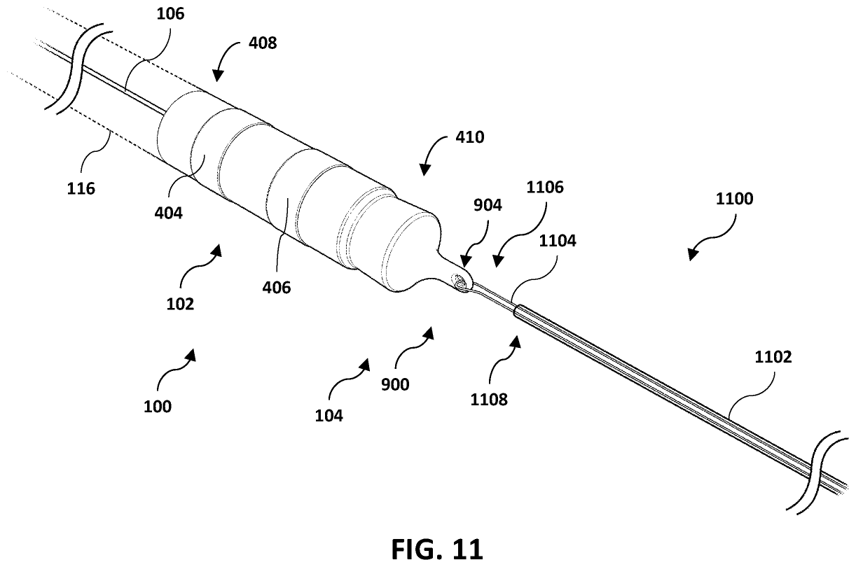

FIG. 11 illustrates the bifunctional connector 100 having the mechanical connector portion 104 provided as the loop connector 900, and a connection catheter 1100 including a catheter 1102 receiving a wire 1104 therethrough, according to one or more embodiment(s). As such, the catheter 1102 and the wire 1104 correspond to the connection medical wire 108. As illustrated, the connection catheter 1100 is connected to the loop connector 900 by the wire 1104 engaging and passing through the hole 904 of the loop connector 900, such that the connection catheter 1100 and the loop connector 900 are in a connected state. In the connected state, an intermediate portion of the cable 1106 is disposed to a connecting end portion 1108 of the catheter 1102 where the wire 1104 passes through the hole 904, while the two end portions (not shown) of the wire 1104 are disposed to an opposed end portion of the catheter (not shown). In FIGS. 9-11, the electrical leads are omitted from the power wire 116 for the sake of clarity.

The connection catheter 1100 and the loop connector 900 may also be in an unconnected state with the wire 1104 not connected to the loop connector 900 and not engaging and passing through the hole 904 of the loop connector 900. To transition from the connected state to the unconnected state, one of the two end portions of the wire 1104 is released from the opposed end portion of the catheter 1102, and the other end portion of the wire 1104 is pulled inside the catheter 1102 to remove the wire 1104 from the hole 904, disconnecting the loop connector 900. Alternatively, the wire 1104 may be cut to transition from the connected state to the unconnected state. Still alternatively, the first and second end portions of the wire 1104 may be tied together, such as at the opposed end portion of the catheter 1102, and the wire 1104 may be cut at the connecting end portion 1108 and/or opposed end portion of the catheter 1102 to release one of the two end portions of the cable, disconnecting the loop connector 900.

With the opposed end portion of the catheter 1102 disposed outside a lumen of the subject body conduit, releasing one of the two end portions of the wire 1104 from the opposed end portion of the catheter 1102 or cutting the wire 1104 may advantageously enables disconnection of the connection catheter 1100 from the loop connector 900 inside a lumen of a subject body conduit.

The connection between the connection catheter 1100 and the loop connector 900 is rotatable since the wire 1104 is capable of twisting on itself inside the catheter 1102 as the connection catheter 1100 is rotated relative to the loop connector 900.

Referring to FIGS. 9-11, it will be appreciated that the electrical connector portion 102 of the bifunctional connector 100 having the loop connector 900 is provided with two peripheral electrical pins 404, 406, similarly to what has been already described herein for the electrical connector portion 102 of the bifunctional connector 100 having the ball connector 400. Therefore, the description of the electrical connector portion 102 provided with two peripheral electrical pins 404, 406 will not be repeated here for the sake of brevity.

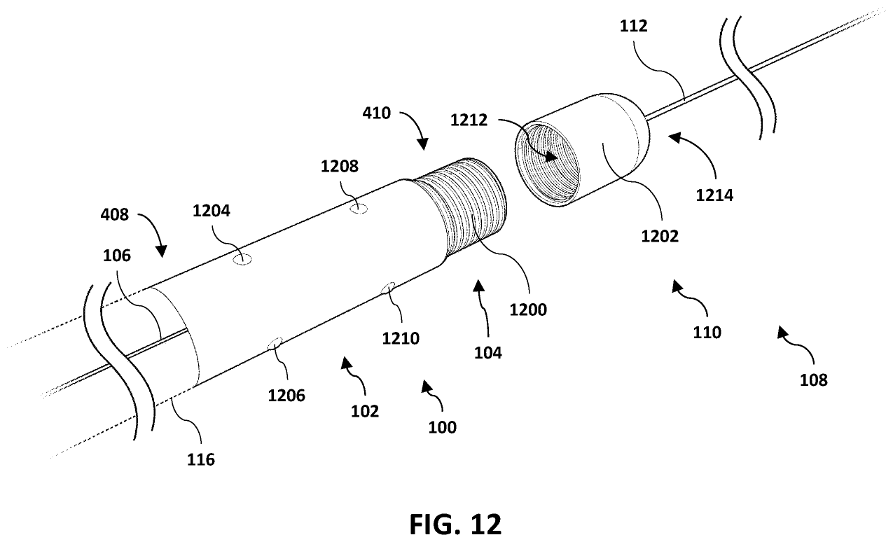

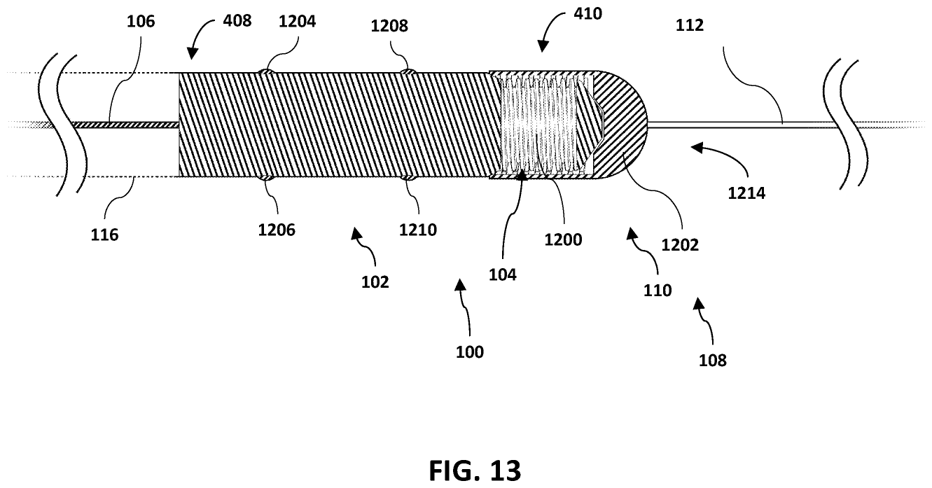

FIGS. 12-13 illustrate the bifunctional connector 100 having the mechanical connector portion 104 provided as a threaded connector 1200, and the connection medical wire 108 having the mechanical connector 110 provided as a correspondent threaded connector 1202 configured to mechanically engage and connect to the threaded connector 1200, according to one or more embodiment(s).

Also illustrated in FIGS. 12-13 is the electrical connector portion 102 having a cylindrical shape and four dot electrical pins 1204, 1206, 1208, 1210 disposed between a power wire-coupling end portion 408 to which the power wire 116 is coupled and a controller-connecting end portion 410 to which the threaded connector 1200 is associated. The power wire 116 may be coupled, either directly or indirectly, to the power wire-coupling end portion 408 notably by being welded, screwed, glued, and/or crimped thereto. FIG. 12 illustrates the threaded connector 1200 and the correspondent threaded connector 1202 not connected to each other and thus in an unconnected state. FIG. 13 illustrates the threaded connector 1200 and the correspondent threaded connector 1202 connected to each other and thus in a connected state. In FIGS. 12-13, the electrical leads are omitted from the power wire 116 for the sake of clarity.

As illustrated in FIGS. 12-13, the threaded connector 1200 and the correspondent threaded connector 1202 of the connection medical wire 108 are of a cylindrical shape. The screw thread of the threaded connector 1200 runs peripherally about the outer periphery of the bifunctional connector 100, while the screw thread of the correspondent threaded connector 1202 runs peripherally inside a cavity 1212 thereof. The cavity 1212 is configured to receive the threaded connector 1200 and at least partially the bifunctional connector 100 therein.

As also illustrated, the medical wire 112 of the connection medical wire 108 projects from a medical wire-attachment end portion 1214 of the correspondent threaded connector 1202 and may be rotatably attached, such as by a captive attachment as described hereinbefore, or non-rotatably attached thereto. The rotatable attachment enabling the advantage(s) described hereinbefore. When rotatably attached to the correspondent threaded connector 1202, the medical wire 112 may be rotated without unscrewing the correspondent threaded connector 1202 from the threaded connector 1200. When non-rotatably attached to the correspondent threaded connector 1202, the medical wire 112 may be rotated for unscrewing the correspondent threaded connector 1202 from the threaded connector 1200, which may be advantageous when the bifunctional connector 100 and the connection medical wire 108 is received inside a lumen of a subject body conduit and are required to be disconnected from each other from outside the subject body by manipulation of the medical wire 112.

The threaded connector 1200 and the correspondent threaded connector 1202 are connected together and disconnected from each other by screwing them together and unscrewing them from each other, respectively.

A locking mechanism (not shown) may be provided to the threaded connector 1200 and/or the correspondent threaded connector 1202 for preventing unwanted disconnection for each other. For example, a set screw configured to abut the threaded connector 1200 may be provided to the correspondent threaded connector 1202 for preventing unwanted unscrewing.

A seal (not shown) may be provided to the threaded connector 1200 and/or the correspondent threaded connector 1202 to provide a fluid thigh engagement therebetween when screwed together.

Alternatively or additionally to the dot electrical pins 1204-1210, the bifunctional connector 100 having the threaded connector 1200 may be provided with protruded electrical pin(s) projecting outwardly from the controller-connecting end portion 410 of the bifunctional connector 100. Still alternatively or additionally to the dot electrical pins 1204-1210, the bifunctional connector 100 having the threaded connector 1200 may be provided with electrical socket(s) (not shown) disposed to and projecting inside the controller-connecting end portion 410 of the bifunctional connector 100. Such disposition to the controller-connecting end portion 410 enables the protruded electrical pin(s) and the electrical socket(s) to be received inside the cavity 1212 when the threaded connector 1200 and the correspondent threaded connector 1202 are connected together. Received inside the cavity 1212, the protruded electrical pin(s) and the electrical socket(s) are advantageously protected from the environmental element, such as fluid like blood, that may be deleterious to the electrical pins(s) and/or electrical socket(s).

The electrical connector portion 102 provided with the dot electrical pins, the socket electrical connectors, and/or the protruded electrical pins being generally similar to the electrical connector portion 102 provided with the peripheral electrical pins 404, 406, the electrical connector portion 102 provided with the dot electrical pins, the socket electrical connectors, and/or the protruded electrical pins will not be further described herein for the sake of brevity. It will be appreciated that a similar description applies between the electrical connector portion 102 provided with the dot electrical pins, the socket electrical connectors, and/or the protruded electrical pins and the electrical connector portion 102 provided with the peripheral electrical pins 404, 406, with the necessary change(s), appreciable to the skilled addressee, having been made, if applicable.

The bifunctional connector 100, including the mechanical connector portion 104 and the electrical connector portion 102 and with or without the optional tensile wire 106 and/or the optional power wire 116, as well as the connection medical wire 108, including the mechanical connector 110 and the medical wire 112, are capable of sustaining without failure a pulling force applied to the connection medical wire 108 for moving the implantable medical device 114 inside the lumen of the subject body conduit, when the mechanical connector portion 104 is mechanically connected to the mechanical connector 110.

The mechanical connector portion 104 of the bifunctional connector 100 and the mechanical connector 110 of the connection medical wire 108 are capable of sustaining without disconnecting from each other a pulling force that is applied to the connection medical wire 108, when the mechanical connector portion 104 is mechanically connected to the mechanical connector 110.

For example, such pulling force may be about between, 1-5 Newton, 1-10 Newton, 1-15 Newton, 1-20 Newton, 1-25 Newton, 1-30 Newton, 1-35 Newton, 1-40 Newton, 1-45 Newton, 1-50 Newton, 1-55 Newton, 1-60 Newton, 1-65 Newton, 1-70 Newton, 1-75 Newton, 1-80 Newton, 1-85 Newton, 1-90 Newton, 1-95 Newton, 1-100 Newton, 100-95 Newton, 100-90 Newton, 100-85 Newton, 100-80 Newton, 100-75 Newton, 100-70 Newton, 100-65 Newton, 100-60 Newton, 100-55 Newton, 100-50 Newton, 100-45 Newton, 100-40 Newton, 100-35 Newton, 100-30 Newton, 100-25 Newton, 100-20 Newton, 100-15 Newton, 100-10 Newton, and 100-5 Newton, inclusive.

Referring back to FIGS. 4-13, embodiments of the bifunctional connector 100 including the tensile wire 106 integrated to the power wire 116 are illustrated. In FIGS. 4-13, the power wire 116 is represented is dashed lines to be able to see the tensile wire 106, which is represented in full lines, therethrough; still, both the tensile wire 106 and the power wire 116 are optional to the bifunctional connector 100. The tensile wire 106 has a first end portion and a second end portion. The first end portion of the tensile wire 106 is couplable to the bifunctional connector 100, such as to the power wire-coupling end portion 408, the mechanical connector portion 104, and/or the electrical connector portion 102 thereof, via a tensile wire coupling. The tensile wire coupling may be achieved notably by welding, screwing, gluing, and/or crimping. The second end portion of the tensile wire 106 is configured to be couplable to the implantable medical device 114 (not shown).

The tensile wire 106 may be an electrical conductor for operating the implantable medical device 114.

The tensile wire 106 advantageously enables a greater pulling force to be applied to the power wire 116 without failure, as compared to a pulling force that is applied to the power wire 116 in absence of the tensile wire 106.

The connection medical wire 108 is configured to be navigated and/or steered inside a lumen of a subject body conduit and also to be manipulated for moving the implantable medical device inside the lumen of the subject body conduit when the mechanical connector is connected to the implantable medical device. In particular, the connection medical wire 108, with or without the medical wire 112, is flexible and/or floppy enough to be navigated and/or steered appropriately in tortuous body conduits, such as one having one or more acute angles. The connection medical wire 108 and/or the medical wire 112 may each have a portion thereof, such as a free end portion opposed to the mechanical connector 110, having a different flexibility and/or floppiness characteristic(s) than the remainder of the connection medical wire 108 and/or the medical wire 112. For example, such portion may be more flexible and/or floppier than the remainder of the connection medical wire 108 and/or the medical wire 112. Although the connection medical wire 108 and/or the medical wire 112 may be flexible and/or floppy enough to be appropriately navigated and/or steered intraluminally, the connection medical wire 108 and/or the medical wire 112 may still be rigid enough to be pushed inside a lumen of a subject body conduit, such as through an intraluminal access.

The medical wire 112 may be made of nitinol, stainless steel, and/or any suitable polymer(s). The medical wire 112 may be coated with a biocompatible material and/or a material that reduces friction with the inner wall of body conduit, such as the intima of the vasculature, or improves lubricity.

The bifunctional connector 100 may be configured to be capturable by a snare inside a lumen of a subject body conduit, such as a lumen of a subject vasculature. For example, the ball connector 400, the latch connector 700, and the hook connector 902 may be captured intraluminally by a snare. When the bifunctional connector 100 is captured by the snare, the snare may be manipulated to move intraluminally the implantable medical device 114 coupled to the bifunctional connector 100.

It will be appreciated that the mechanical connector portion 104 of the bifunctional connector 100 and the mechanical connector 110 of the connection medical wire 108 are connectable together independently of the electrical connector portion 102 of the bifunctional connector 100. In particular, the ball connector 400, the latch connector 700, the loop connector 900, and the threaded connector 1200 are connectable to the corresponding the socket connector 402, catch connector 702, hook connector 902 and connection catheter 1100, and the correspondent threaded connector 1202 independently of the electrical engagement and connection to the electrical connector portion 102.

Further, the ball connector 400, the latch connector 700, the loop connector 900, and the threaded connector 1200 are connectable to the corresponding socket connector 402, catch connector 702, hook connector 902 and connection catheter 1100, and the correspondent threaded connector 1202 only when the electrical connector portion 102 is in an unconnected state, e.g., when the electrical connector portion 102 is not connected to the controller electrical connector.

On the other hand, the electrical connector portion 102 may be connectable to the controller electrical connector when the ball connector 400, the latch connector 700, the loop connector 900, and the threaded connector 1200 are disconnected from the corresponding socket connector 402, the catch connector 702, the hook connector 902 and connection catheter 1100, and the correspondent threaded connector 1202, and are thus in an unconnected state. However, the electrical connector portion 102 may be connectable to the controller electrical connector when the ball connector 400, the latch connector 700, the loop connector 900, and the threaded connector 1200 are connected to the corresponding socket connector 402, catch connector 702, hook connector 902 and connection catheter 1100, and the correspondent threaded connector 1202, and are thus in a connected state. In this last case, the connection medical wire 108 may be required to be slid through the controller electrical connector for an appropriate electrical connection of the electrical connector portion 102 to the controller electrical connector.

It will also be appreciated that the mechanical connector portion 104 of the bifunctional connector 100 and the mechanical connector 110 of the connection medical wire 108 are reconnectable together and thus suitable for reuse purpose, which may be advantageous depending on the transcatheter, surgical, and/or medical procedure(s) performed.

It will further be appreciated that any combination(s) of the ball connector 400, socket connector 402, latch connector 700, catch connector 702, hook connector 902, loop connector 900, connection catheter 1100, threaded connector 1200, correspondent threaded connector 1202, peripheral electrical pins, longitudinal electrical pins, dot electrical pins, protruded electrical pins, and electrical sockets are encompassed herein without departing from the scope of this disclosure, with the necessary change(s), appreciable to the skilled addressee, having been made, if applicable. In particular, any embodiment(s) of the mechanical connector portion 104 disclosed herein may be combined with any embodiment(s) of the electrical connector portion 102 disclosed herein.

The connection medical wire 108 may include a disconnection mechanism (not shown) configured to disconnect the mechanical connector 110 thereof from the mechanical connector portion 104 of the bifunctional connector 100. For example, the gate member of the hook connector 902 may be coupled to and actuated by an elongated actuation member of the disconnection mechanism running along the medical wire 112. The elongated actuation member may be manipulable at an end portion of the medical wire 112 that is opposed to the end portion of the medical wire 112 attaching the hook connector 902. Upon actuation, the disconnection mechanism actuates the gate member from the close position to the open position. In the open position, the medical wire 112 may be manipulated, such as by being pushed toward the loop connector 900, to disconnect the hook connector 902 from the loop connector 900. The disconnection mechanism may be useful for disconnecting the mechanical connector 110 and the mechanical connector portion 104 form each other inside a lumen of a subject body conduit, such as a lumen of a subject vasculature, from outside the subject body by manipulation of the elongated actuation member.

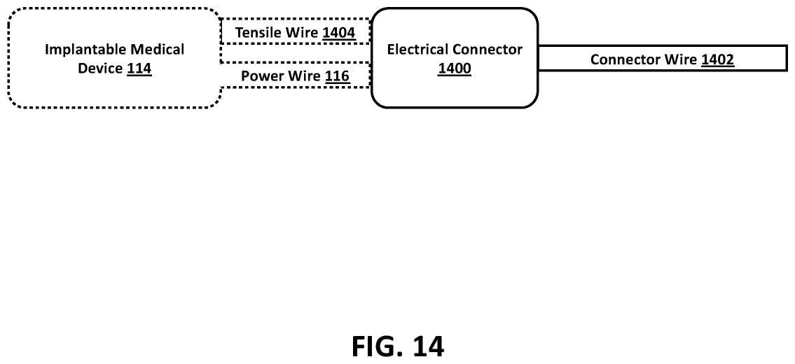

Turning now to FIGS. 14-17, this disclosure also relates generally to an electrical connector 1400 (also referred to herein as an “electrical connector portion 1400”) for the optional implantable medical device 114 and includes a connector wire 1402 (also referred to herein as a “wire 1402”), the optional power wire 116, and an optional tensile wire 1404, according to a third aspect of the disclosed technology. When projecting from the electrical connector 1400 provided to the optional power wire 106, the connector wire 1402 may generally increase the useful length and reach of the optional power wire 116 for in vivo application, as described for the connection medical wire 108. For example, the connector wire 1402 may be manipulable by an operator to move the optional implantable medical device 114 inside a lumen of a subject body conduit. The electrical connector 1400 may also establish an electrical connection between the optional implantable medical device 114 and an optional controller 1500 for operating the optional implantable medical device 114 by the optional controller 1500 through the electrical connector 1400.

In particular, FIG. 14 schematically represents the electrical connector 1400 in an electrically unconnected state, according to one or more embodiment(s). In the electrically unconnected state, the electrical connector 1400 has the connector wire 1402 projecting therefrom and is electrically unconnected from a controller (not shown).

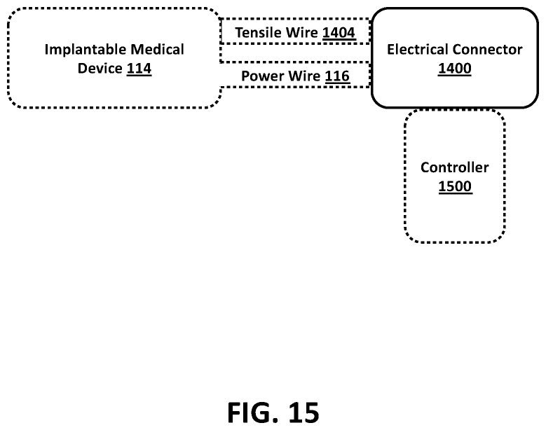

FIG. 15 schematically represents the electrical connector 1400 in an electrically connected state, according to one or more embodiment(s). In the electrically connected state, the electrical connector 1400 is electrically connected to the optional controller 1500 for electrically operating the optional implantable medical device 114 through the electrical connector 1400 by the controller 1500. Still in the electrically connected state, the connector wire 1402 may be removed from the electrical connector 1400 (as shown), such as by being cut therefrom, for electrical connection between the electrical connector 1400 and the optional controller 1500. Alternatively, the connector wire 1402 may be folded along the optional power wire 116 or may be passed through a corresponding electrical connector of the optional controller 1500 for electrical connection between the electrical connector 1400 and the optional controller 1500.

Throughout FIGS. 14-15, the optional tensile wire 1404 and the optional power wire 116 are represented as separate structures, but they may be integrated together as illustrated in one or more of the embodiment(s) described hereinafter.

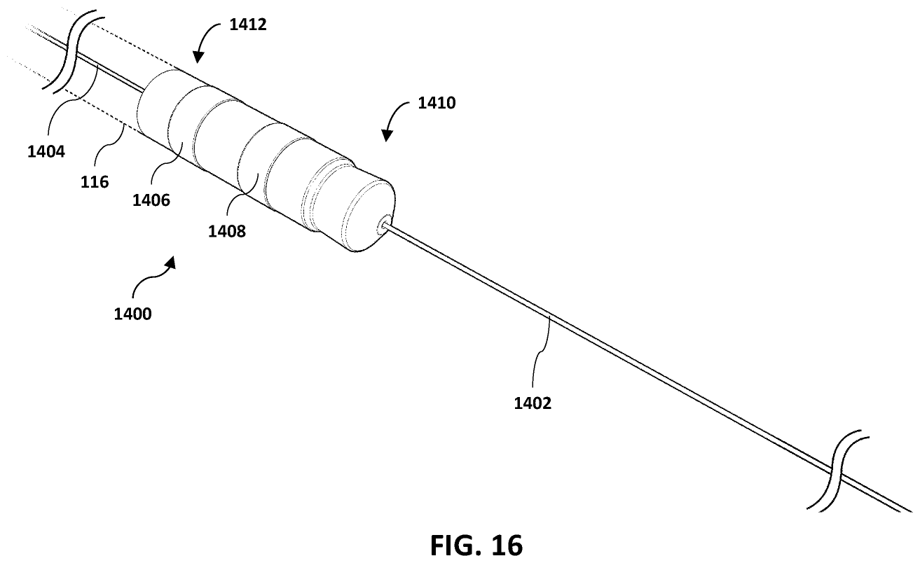

FIGS. 16-18 illustrate the electrical connector 1400 having a body of a cylindrical shape and two peripheral electrical pins 1406, 1408 disposed between a controller-connecting end portion 1410 and a power wire-coupling end portion 1412 to which the optional tensile wire 1404 and the optional power wire 116 are coupled thereto, according to one or more embodiment(s). The power wire 116 may be coupled, either directly or indirectly, to the power wire-coupling end portion 408 notably by being welded, screwed, glued, and/or crimped thereto. The tensile wire 1404 enables the same advantage(s) as described hereinbefore. In FIG. 16-18, the power wire 116 is represented is dashed lines to be able to see the tensile wire 1404, which is represented in full lines, therethrough; still, both the tensile wire 1404 and the power wire 116 are optional to the electrical connector 1400. In FIGS. 16-18, the electrical leads are omitted from the power wire 116 for the sake of clarity.

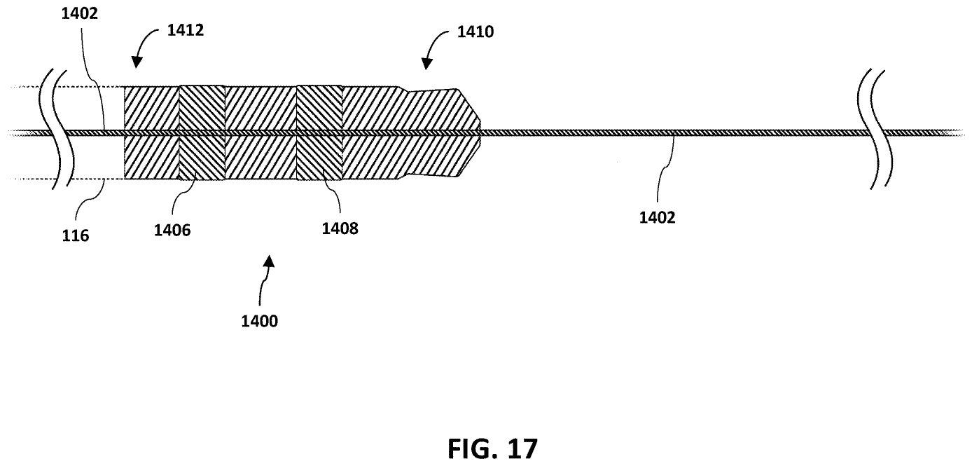

FIGS. 16-17 illustrate the electrical connector 1400 including the connector wire 1402 that projects coaxially from the controller-connecting end portion 1410, according to one or more embodiment(s). Alternatively, the connector wire 1402 may project non-coaxially and/or may project at an angle from the controller-connecting end portion 1410.

In particular, FIG. 16 illustrates the optional tensile wire 1404 that projects in a direction opposite to the connector wire 1402 and in the same direction as the optional power wire 116. The connector wire 1402 is coupled, either directly or indirectly, to the controller-connecting end portion 1410 of the electrical connector 1400, such as by being welded, screwed, glued, and/or crimped thereto. Alternatively, the connector wire 1402 may form an integral, one-piece construction with the electrical connector 1400. The optional tensile wire 1404 and/or the optional power wire 116 may be coupled to the power wire-coupling end portion 1412 of the electrical connector 1400, such as by being welded, screwed, glued, and/or crimped thereto. Alternatively, the optional tensile wire 1404 and/or the optional power wire 116 may form an integral, one-piece construction with the connector wire 1402.

The connector wire 1402 may be rotatably attached, such as by a captive attachment as described hereinbefore, or non-rotatably attached to the controller-connecting end portion 1410 of the electrical connector 1400. The rotatable attachment enables the same advantage(s) as described hereinbefore.

FIG. 17 illustrates the connector wire 1402 coupled to the body of the electrical connector 1400, extending therealong, and projecting outwardly therefrom from in opposite directions, i.e. away from the controller-connecting end portion 1410 and away from the power wire-coupling end portion 1412 (not shown), according to one or more embodiment(s). As such, the connector wire 1402 may project coaxially or non-coaxially from the controller-connecting end portion 1410 and/or the power wire-coupling end portion 1412 of the electrical connector 1400. The connector wire 1402 may also project at an angle or not from the controller-connecting end portion 1410 and/or the power wire-coupling end portion 1412 of the electrical connector 1400. Therefore, it will be appreciated that the connector wire 1402 illustrated in FIG. 17 and described herein may be replaced by the tensile wire 1404, and vice versa, for the same purpose.

Advantageously, having no couplings between the connector wire 1402 and the electrical connector 1400 as well as between the tensile wire 1404 and the electrical connector 1400, the electrical connector 1400 of FIG. 17 provides for a construction having fewer failure modes than the electrical connector 1400 of FIG. 16. Indeed, depending on the pulling force applied to the connector wire 1402 of the electrical connector of FIG. 16, one or two of the coupling(s) may break, whereas such failure mode is absent from the electrical connector of FIG. 17.

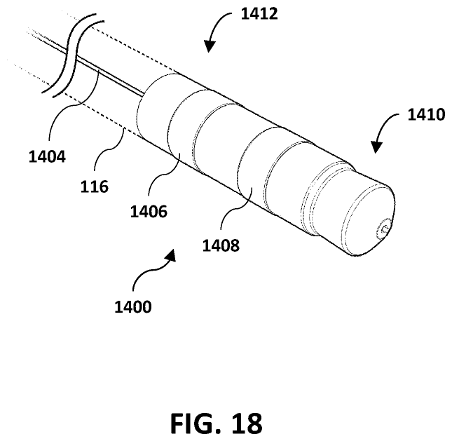

FIG. 18 illustrates the electrical connector 1400 having the connector wire 1402 removed therefrom and ready for being connected to a controller (not shown) for operating the optional implantable medical device 114. In particular, the connector wire 1402 may be removed from the electrical connector 1400 by being cut therefrom by a cutting tool (not shown), such as a cutting pliers. As illustrated, the connector wire 1402 has been cut close to the controller-connecting end portion 1410; however, the connector wire 1402 is cuttable anywhere along its length such that a portion of the connector wire 1402 may remain thereafter. If not cut or if a portion remains, the connector wire 1402 may be required to be slid through the controller electrical connector for an appropriate electrical connection of the electrical connector portion 102 to the controller electrical connector.

The electrical connector 1400, including the connector wire 1402 and with or without the optional tensile wire 1404 and/or the optional power wire 116, is capable of sustaining without failure a pulling force applied to the connector wire 1402 for moving the implantable medical device 114 inside the lumen of the subject body conduit, when the electrical connector 1400 is coupled to the implantable medical device 114. The pulling force is the same as the pulling forces described hereinbefore.

The electrical connector 1400 being generally similar to the electrical connector portion 102 of FIGS. 4-6, the electrical connector 1400 will not be further described herein for the sake of brevity. It will be appreciated that a similar description applies between the electrical connector portion 102 of FIGS. 4-6 and the electrical connector 1400, with the necessary change(s), appreciable to the skilled addressee, having been made, if applicable.

Although the electrical connector 1400 is illustrated with the peripheral electrical pins 1406, 1408, which generally correspond to the peripheral electrical pins 404, 406, the electrical connector 1400 may be provided with any type of electrical pins or contacts, such as longitudinal electrical pins or dot electrical pins, as described hereinbefore, with the necessary change(s), appreciable to the skilled addressee, having been made, if applicable.

It will be appreciated that the electrical connector 1400 may be suitable for single-use purpose in which the connector wire 1402 may be discarded after removal from the electrical connector 1400.

Although the electrical connector portion 102 and the electrical connector 1400 are illustrated herein as having a cylindrical shape, these components may have any other appropriate shapes, such as an oval shape or an ovaloid shape.

The bifunctional connector 100, the mechanical connector 110 of the connection medical wire 108, and the electrical connector 1400 are sized to be received inside the lumen of the subject body conduit, such as a lumen of a subject vasculature. In particular, the bifunctional connector 100, the mechanical connector 110, and the electrical connector 1400 may have a cross-sectional size of about between 1 FR and 40 FR, inclusively. For example, such cross-sectional sizes may be about between 1-5 FR, 1-10 FR, 1-15 FR, 1-20 FR, 1-25 FR, 1-30 FR, 1-35 FR, 1-40 FR, 40-35 FR, 40-30 FR, 40-25 FR, 40-20 FR, 40-15 FR, 40-10 FR, 40-5 FR, 40-1 FR, 1-5 FR, 6-10 FR, 11-15 FR, 16-20 FR, 21-25 FR, 26-30 FR, 31-35 FR, and 36-40 FR, inclusive. The bifunctional connector 100, the mechanical connector 110, and the electrical connector 1400 may each have the same, substantially the same, or different cross-sectional size(s).

In application where a lumen of a catheter, or other similar tubular structure, is in fluid communication with blood presents in a subject vasculature, the bifunctional connector 100 (and the power wire 116 of the implantable medical device 114 when coupled thereto) and the connection medical wire 108 may be so cross-sectionally sized and shaped that they seal the catheter's lumen and may be slid inside the catheter's lumen a fluid-thigh manner without blood coming out from the catheter's lumen.

The bifunctional connector 100 and the connection medical wire 108 may be used advantageously, alone or in combination, with a power wire of an implantable medical device for extending the length of the power wire to perform some transcatheter, surgical, and/or other medical procedure(s). Such power wire length extension may be momentarily to enables navigation of the power wire and movement of the implantable medical device inside a lumen of a subject body conduit. Afterward, the length of the power wire may be reduced by disconnecting the connection medical wire 108 therefrom, and the power wire may be connected to a controller, via the bifunctional connector 100, for operating the implantable medical device inside the lumen of the subject body conduit.

The bifunctional connector 100 and the tensile wire 106 may be provided as a kit.

The bifunctional connector 100, with or without the tensile wire 106, and the connection medical wire 108 may be provided as a kit.

A power wire including the bifunctional connector 100, with or without the tensile wire 106, may be provided as a kit.

A power wire including the bifunctional connector 100, with or without the tensile wire 106, and the connection medical wire 108 may be provided as a kit.

An implantable medical device including the bifunctional connector 100, with or without the tensile wire 106, may be provided as a kit.

An implantable medical device including the bifunctional connector 100, with or without the tensile wire 106, and the connection medical wire 108 may be provided as a kit.

An implantable medical device including the bifunctional connector 100, with or without the tensile wire 106, and the connection medical wire 108 may be provided as a kit.

The electrical connector 1400 and the tensile wire 1404 may be provided as a kit.

The electrical connector 1400, with or without the tensile wire 1404, and the connector wire 1402 may be provided as a kit.

A power wire including the electrical connector 1400, with or without the tensile wire 1404, may be provided as a kit.

A power wire including the electrical connector 1400, with or without the tensile wire 1404, and the connector wire 1402 may be provided as a kit.

An implantable medical device including the electrical connector 1400, with or without the tensile wire 1404, may be provided as a kit.

An implantable medical device including the electrical connector 1400, with or without the tensile wire 1404, and the connector wire 1402 may be provided as a kit.

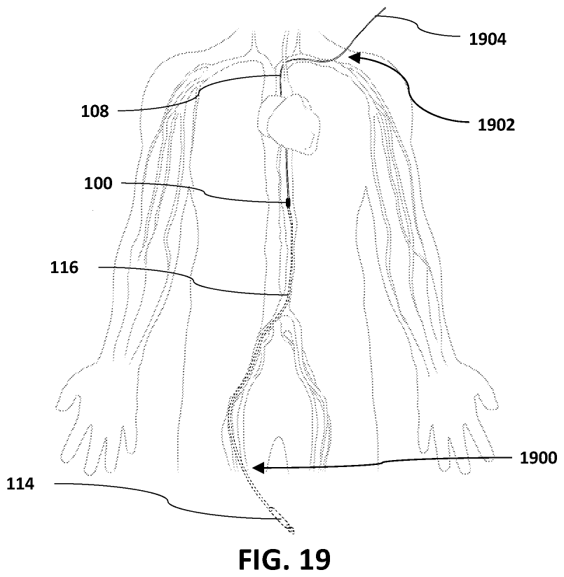

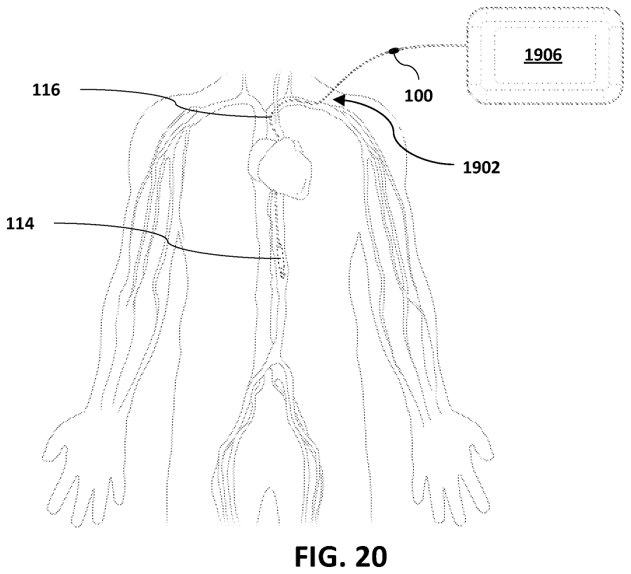

FIGS. 19-20 illustrate a transcatheter cardiovascular application of the bifunctional connector 100 used in combination with the connection medical wire 108, as an example of how the technology can be implemented in vivo, according to one or more embodiment(s). In particular, FIG. 19 illustrates the bifunctional connector 100 being provided to a power wire 116, which may or may not include the tensile wire 116, coupled to an implantable medical device 114. The mechanical connector portion 104 of the bifunctional connector 100 is mechanically connected to the mechanical connector 110 of the connection medical wire 108.

As illustrated, the connection medical wire 108 and the power wire 116 are collectively long enough to have been routed from a first subject intraluminal access 1900 to a second subject intraluminal access 1902. Alternatively, the connection medical wire 108 may be long enough to be routed from the first subject intraluminal access 1900 to a second subject intraluminal access 1902, such that the power wire 116 is not yet routed through the first intraluminal access 1900.

For example, to implant the implantable medical device 114 at an intraluminal implantation site of the subject, a leading end portion 1904 of the connection medical wire 108 is first introduced through the first subject intraluminal access 1900, advanced along the lumen of the subject vasculature, and externalized through the second subject intraluminal access 1902. Then, the leading end portion 1904 of the connection medical wire 108, which at this point is disposed outside the lumen of the subject vasculature, is pulled through the second subject intraluminal access 1902. Alternatively or additionally, the connection medical wire 108, the power wire 116, and/or the implantable medical device 114 may be pushed through the first subject intraluminal access 1900. Doing so, the implantable medical device 114 is moved through the first subject intraluminal access 1900 and inside the lumen of the subject vasculature up to the intraluminal implantation site thereof, and the power wire 116 is externalized through the second subject intraluminal access 1902 for electrical connection to a controller.

FIG. 20 illustrates the implantable medical device 114 positioned at the intraluminal implantation site with the power wire 116 thereof routed therefrom, along the lumen of the subject vasculature, up to and through the second subject intraluminal access 1902. The connection medical wire 108, which is absent from FIG. 20, has been disconnected and removed from the bifunctional connector 100, which is now electrically connected to a controller 1906 for operating the implantable medical device 114 inside the lumen of the subject vasculature.

It will be appreciated from FIGS. 19-20 that the bifunctional connector 100 and the connection medical wire 108 are advantageous when the power wire 116 is not long enough in itself to be appropriately routed from the first subject intraluminal access to the second subject intraluminal access and externalized therethrough. In contrast, the bifunctional connector 100 connected to the connection medical wire 108 are collectively long enough to be routed from the first subject intraluminal access to the second subject intraluminal access and externalized therethrough, such that the implantable medical device 114 may be connected to the controller 1906 to be operated, as illustrated in FIGS. 19-20.

The bifunctional connector 100 and/or the connection medical wire 108 advantageously enables the length of a power wire of an implantable medical device to be increased and reduced at some point during transcatheter, surgical, and/or other medical procedure(s), depending on the operator's need, to avoid manipulating an unnecessary long power wire. Indeed, the power wire length modulation enabled by the bifunctional connector 100 and/or the connection medical wire 108 advantageously avoid the need, once an implantable medical device is implanted to a subject, to manage an unnecessary long or short power wire by, for example, tunneling at least a portion of the power wire under the skin or creating a large subcutaneous pocket to contain at least a portion of an unnecessarily long power wire therein.

The connection medical wire 108 advantageously facilitates intraluminal navigation of a power wire that is not necessarily configured to be routed and/or steered inside a lumen of a subject body conduit. Indeed, with the connection medical wire 108 connected to such a power wire, the medical wire 112 may add a flexible or floppy, or more flexible or floppier as compared to the power wire, extremity to the power wire that facilitates intraluminal routing and/or steerability. This enables the power wire to be routed through tortuous vascular anatomy or vascular anatomy having one or more acute angles where the power wire cannot or cannot easily pass through in absence of the medical wire 112. This also enables the power wire to be intraluminally captured or to be more easily intraluminally captured by snaring the connection medical wire 108.

The connection medical wire 108 may be used alone and be connected to any implantable medical device for routing a power wire thereof and/or moving the implantable medical device inside a lumen of the subject body conduit. In this case, the implantable medical device has a connector that is compatible with the mechanical connector 110 of the connection medical wire 108. Similarly, any implantable medical device provided with a bifunctional connector 100 may be used alone and be connected to a wire, cable, or other corresponding structure for routing a power wire thereof and/or moving the implantable medical device inside a lumen of the subject body conduit. In this case, the wire, cable, or other corresponding structure has a connector that is compatible with the mechanical connector portion 104 of the bifunctional connector 100.

It will be appreciated that the transcatheter cardiovascular application illustrated in FIGS. 19-20 and described herein may also be performed using the electrical connector 1400 by cutting the connector wire 1402 therefrom instead of disconnecting the connection medical wire 108 from the mechanical connector portion 104 of the bifunctional connector 100, with the necessary change(s), appreciable to the skilled addressee, having been made, if applicable.

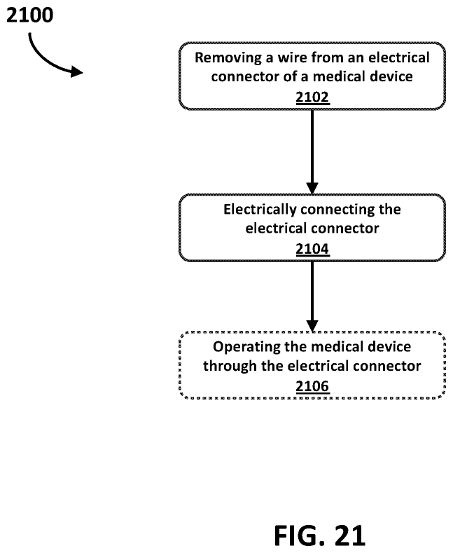

Turning now to FIG. 21, this disclosure also relates generally to a method 2100 of using an electrical connector (e.g., the bifunctional connector 100 and the electrical connector 1400) of a medical device, according to a fourth aspect of the disclosed technology. Element(s) and/or feature(s) that are optional to the method 2100 are represented in dashed lines in FIG. 21. It will be noted that references to the bifunctional connector 100, the connection medical wire 108, the electrical connector 1400, the connector wire 1402, and/or any other element(s) or features(s) thereof, as the case may be, are made as example(s) and for the sole purpose of describing how the method 2100 can be implemented. Such references are not intended in any way to limit the scope of the method 2100.

As schematically represented in FIG. 21, the method 2100 includes: removing, at 2102 a wire (e.g., the connection medical wire 108, the connector wire 1402, a guide wire, a cable, a lead, and an elongated structure) from an electrical connector (e.g., the electrical connector portion 102 and the electrical connector 1400) of a medical device (e.g., the implantable medical device 114); and electrically connecting, at 2104, the electrical connector, according to one or more embodiment(s). Removing, at 2102, may be performed before electrically connecting, at 2104.

Removing, at 2102, may include disconnecting the wire and the electrical connector from each other. For example, the electrical connector may be an electrical connector portion (e.g., the electrical connector portion 102), and the electrical connector may include a mechanical connector portion (e.g., the mechanical connector portion 104). In this case, removing, at 2102, may include disconnecting the mechanical connector of the wire and the mechanical connector portion of the electrical connector from each other to remove the wire from the electrical connector.

Disconnecting the mechanical connector of the wire and the mechanical connector portion of the electrical connector from each other may include disconnecting at least one of: (i) a ball connector (e.g., the ball connector 400) and a socket connector (e.g., the socket connector 402) of the wire from a corresponding one of a socket connector (e.g., the socket connector 402) and a ball connector (e.g., the ball connector 400) of the medical device; (ii) a catch connector (e.g., the catch connector 702) and a latch connector (e.g., the latch connector 700) of the wire from a corresponding one of a latch connector (e.g., the latch connector 700) and a catch connector (e.g., the catch connector 702) of the medical device; (iii) a hook connector (e.g., the hook connector 902) and a loop connector (e.g., the loop connector 900) of the wire from a corresponding one of a loop connector (e.g., the loop connector 900) and a hook connector (e.g., the hook connector 902) of the medical device; (iv) a wire (e.g., the wire 1104 of the catheter 1102 of the connection catheter 1100) from a loop connector (e.g., the loop connector 900) of the medical device; and (v) a threaded connector of the wire from a correspondent threaded connector of the medical device (e.g., the threaded connector 1200 and the correspondent threaded connector 1202).

Removing, at 2102, may include cutting the wire (e.g., the connector wire 1402, the connection medical wire 108, and the medical wire 112) to remove at least a portion thereof from the electrical connector (e.g., the electrical connector 1400).

Electrically connecting, at 2104, may include electrically connecting the electrical connector to a controller (e.g. the controllers 300, 1500) configured to electrically operate the medical device through the electrical connector.

Electrically connecting, at 2104, may include electrically connecting the electrical connector provided to a power wire (e.g., the power wire 116, which may or may not include the tensile wire 106) of the medical device.

According to the method 2100, instead of removing, at 2102, the wire, the method 2100 may alternatively include: passing the wire (e.g., the connector wire 1402) through an electrical connector of the medical device to connect the electrical connector to a corresponding electrical connector of a controller configured to operate the medical device. Still alternatively, the method 2100 may include: folding the wire (e.g., the connector wire 1402) to connect the electrical connector to a corresponding electrical connector of a controller configured to operate the medical device.

The method 2100 may include: electrically operating, at 2106, the medical device through the electrical connector (e.g., the bifunctional connector 100 and the electrical connector 1400). For example, the medical device may be electrically operated inside a subject body through the electrical connector by a controller (e.g. the controllers 300, 1500) located outside the subject body. Electrically operating, at 2106, the medical device may be performed after electrically connecting, at 2104.

The method 2100 may include: obtaining a first subject intraluminal access (e.g., first subject intraluminal access 1900) and a second subject intraluminal access (second subject intraluminal access 1902). Depending on the medical procedure, the first and/or second subject intraluminal access(es) may be a surgically made intraluminal access or a natural intraluminal access. Obtaining a first subject intraluminal access and a second subject intraluminal access may be performed before removing, at 2102. Each of the first and second subject intraluminal accesses may be a lower subject body intraluminal accesses and/or an upper subject body intraluminal accesses. For example, the lower subject body intraluminal accesses may be a femoral access, and the upper subject body intraluminal accesses may be an axillary or subclavian access.

The method 2100 may include: introducing a leading end portion of the wire through the first subject intraluminal access; routing the leading end portion of the wire inside a lumen of a subject body conduit up to the second subject intraluminal access; and externalizing the leading end portion of the wire through the second subject intraluminal access (in this case, the leading end portion of the wire may also be referred to herein as an “extraluminally disposed end portion” of the wire). As such, the wire and the power wire of the medical device connected to the wire may be configured for a transcatheter application. For example, the wire and the power wire of the medical device may be configured to be passed through one or more subject intraluminal accesses and to be routed inside a lumen of a subject body conduit. For example, the leading end portion of the wire may correspond to a free end portion of the connection medical wire 108 that is opposed to the mechanical connector 110 or to a free end portion of the connector wire 1402 that is opposed to the electrical connector 1400. Such a free end portion is illustrated in FIGS. 19-20. Introducing a leading end portion of the wire, routing the leading end portion of the wire, and externalizing the leading end portion of the wire may all be performed before removing, at 2102.

The method 2100 may include: manipulating the extraluminally disposed end portion of the wire externalized at the second subject intraluminal access. Manipulating the extraluminally disposed end portion of the wire may be performed after externalizing the leading end portion of the wire through the second subject intraluminal access.

Manipulating the extraluminally disposed end portion of the wire may include pulling the extraluminally disposed end portion of the wire through the second subject intraluminal access to introduce the medical device through the first subject intraluminal access inside a lumen of a subject body conduit and/or to move the medical device inside a lumen of a subject body conduit, for example, up to an intraluminal implantation site.

Manipulating the extraluminally disposed end portion of the wire may include applying to the extraluminally disposed end portion of the wire a pulling force between about 1-2 Newton, 1-3 Newton, 1-4 Newton, 1-5 Newton, 1-6 Newton, 1-7 Newton, 1-8 Newton, 1-9 Newton, 1-10 Newton, 10-9 Newton, 10-8 Newton, 10-7 Newton, 10-6 Newton, 10-5 Newton, 10-4 Newton, 10-3 Newton, 10-2 Newton, 10-1 Newton, 1-2 Newton, 2-4 Newton, 6-8 Newton, and 9-10 Newton, inclusive.

Manipulating the extraluminally disposed end portion of the wire may include pushing the extraluminally disposed end portion of the wire through the second subject intraluminal access to move the medical device inside a lumen of a subject body conduit, for example, to move the medical device from an intraluminal implantation site.

The method 2100 may include: manipulating the wire at the first subject intraluminal access. Manipulating the wire at the first subject intraluminal access may be performed after introducing a leading end portion of the wire through the first subject intraluminal access.

Manipulating the wire at the first subject intraluminal access may include pushing the wire through the first subject intraluminal access inside a lumen of a subject body conduit, for example, to route the wire therein.

The method 2100 may include: removably connecting the wire to the electrical connector of the medical device (e.g., by removably connecting the mechanical connector 110 of the connection medical wire 108 to the mechanical connector portion 104 of the bifunctional connector 100). For example, the wire may be removably connected to the electrical connector provided to a power wire (e.g., the power wire 116, which may or may not include the tensile wire 106) of the medical device and configured to operate the medical device therethrough by a controller. Removably connecting the wire to the electrical connector of the medical device may be performed before manipulating the extraluminally disposed end portion of the wire.

Removably connecting the wire to the electrical connector of the medical device may include removably connecting the wire to the electrical connector of the medical device independently of any electrical connection of the electrical connector (e.g., via the electrical connector portion 102 of the bifunctional connector 100) used for electrical operation of the medical device.

Removably connecting the wire to the electrical connector of the medical device may include removably connecting the wire to the electrical connector of the medical device only when the electrical connector is in an electrically unconnected state (e.g., when the electrical connector portion 102 is not connected).

Removably connecting the wire to the electrical connector of the medical device may include removably connecting at least one of: (i) a ball connector (e.g., the ball connector 400) and a socket connector (e.g., the socket connector 402) of the wire to a corresponding one of a socket connector (e.g., the socket connector 402) and a ball connector (e.g., the ball connector 400) of the medical device; (ii) a catch connector (e.g., the catch connector 702) and a latch connector (e.g., the latch connector 700) of the wire to a corresponding one of a latch connector (e.g., the latch connector 700) and a catch connector (e.g., the catch connector 702) of the medical device; (iii) a hook connector (e.g., the hook connector 902) and a loop connector (e.g., the loop connector 900) of the wire to a corresponding one of a loop connector (e.g., the loop connector 900) and a hook connector (e.g., the hook connector 902) of the medical device; (iv) a wire (e.g., the wire 1104 of the catheter 1102 of the connection catheter 1100) to a loop connector (e.g., the loop connector 900) of the medical device; and (v) a threaded connector of the wire to a correspondent threaded connector of the medical device (e.g., the threaded connector 1200 and the correspondent threaded connector 1202).

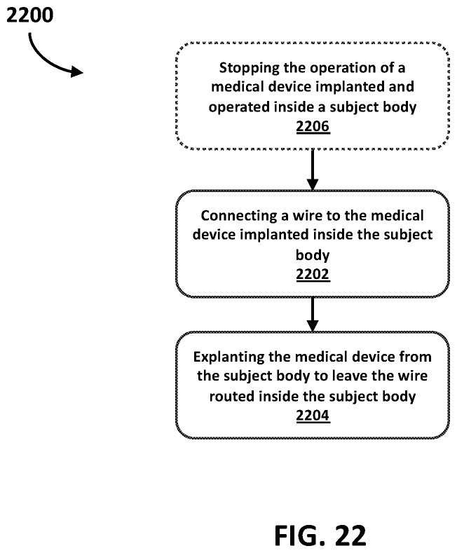

Turning now to FIG. 22, this disclosure also relates generally to a method 2200 of using an electrical connector (e.g., the bifunctional connector 100) of a medical device implanted in a subject body (also referred to herein as an “implanted medical device”), according to a fifth aspect of the disclosed technology. Element(s) and/or feature(s) that are optional to the method 2200 are represented in dashed lines in FIG. 22. It will be noted that references to the bifunctional connector 100, the connection medical wire 108, and/or any other element(s) or features(s) thereof, as the case may be, are made as example(s) and for the sole purpose of describing how the method 2200 can be implemented. Such references are not intended in any way to limit the scope of the method 2200.

As schematically represented in FIG. 22, the method 2200 includes: connecting, at 2202, a wire (e.g., the connection medical wire 108, a guide wire, a cable, a lead, and an elongated structure) to an implanted medical device (e.g., to the mechanical connector portion 104 of the bifunctional connector 100 provided to the power wire 116 of the implantable medical device 114); and explanting, at 2204, the implanted medical device from the subject body to leave the wire routed inside the subject body, according to one or more embodiment(s). Connecting, at 2202, may be performed before explanting, at 2204.

Connecting, at 2202, may include: connecting the wire to an electrical connector of the implanted medical device.

Connecting, at 2202, may include: removably connecting the wire to the implanted medical device.

Removably connecting the wire to the implanted medical device may include removably connecting the wire to an electrical connector (e.g., the bifunctional connector 100) of the implanted medical device independently of any electrical connection of the electrical connector (e.g., via the electrical connector portion 102 of the bifunctional connector 100) used for electrical operation of the implanted medical device.

Removably connecting the wire to implanted medical device may include removably connecting the wire to an electrical connector (e.g., the bifunctional connector 100) of the implanted medical device only when the electrical connector is in an electrically unconnected state (e.g., when the electrical connector portion 102 is not connected).

Removably connecting the wire to the implanted medical device may include removably connecting at least one of: (i) a ball connector (e.g., the ball connector 400) and a socket connector (e.g., the socket connector 402) of the wire to a corresponding one of a socket connector (e.g., the socket connector 402) and a ball connector (e.g., the ball connector 400) of the implanted medical device; (ii) a catch connector (e.g., the catch connector 702) and a latch connector (e.g., the latch connector 700) of the wire to a corresponding one of a latch connector (e.g., the latch connector 700) and a catch connector (e.g., the catch connector 702) of the implanted medical device; (iii) a hook connector (e.g., the hook connector 902) and a loop connector (e.g., the loop connector 900) of the wire to a corresponding one of a loop connector (e.g., the loop connector 900) and a hook connector (e.g., the hook connector 902) of the implanted medical device; (iv) a wire (e.g., the wire 1104 of the catheter 1102 of the connection catheter 1100) to a loop connector (e.g., the loop connector 900) of the implanted medical device; and (v) a threaded connector of the wire to a correspondent threaded connector of the implanted medical device (e.g., the threaded connector 1200 and the correspondent threaded connector 1202).

Explanting, at 2204, may include transcatheterly explanting the implanted medical device.

Explanting, at 2204, may include snaring the implanted medical device from a subject intraluminal access (e.g., the first subject intraluminal access 1900) and pulling the snare through the subject intraluminal access to move the implanted medical device inside the subject body, to internalize through another subject intraluminal access (e.g., the second subject intraluminal access 1902) a portion of the medical device (e.g., the power wire 116 provided with the bifunctional connector 100; also referred to herein as an “externalizable and connectable end portion” of the medical device) connected to the wire and/or the wire, and to leave the wire routed between the other subject intraluminal access (e.g., the second subject intraluminal access 1902) and the subject intraluminal access (e.g., the first subject intraluminal access 1900) inside the subject body.

Explanting, at 2204, may include pushing the externalizable and connectable end portion of the medical device through the other subject intraluminal access (e.g., the second subject intraluminal access 1902) to move the implanted medical device inside the subject body. Pushing he externalizable and connectable end portion of the medical device may be performed before explanting, at 2204.

The method 2200 may include: obtaining the subject intraluminal access (e.g., the first subject intraluminal access 1900) to introduce the snare therethrough to explant the implanted medical device. Obtaining the subject intraluminal may be performed before explanting, at 2204. Each of the subject intraluminal access and other subject intraluminal access may be a lower subject body intraluminal accesses and/or an upper subject body intraluminal accesses. For example, the lower subject body intraluminal accesses may be a femoral access, and the upper subject body intraluminal accesses may be an axillary or subclavian access.

The method 2200 may include: stopping, at 2206, the operation of the implanted medical device being operated. For example, the implanted medical device may be operated inside the subject body, such by through an electrical connector (e.g., the bifunctional connector 100) by a controller (e.g. the controllers 300, 1500) located outside the subject body. Stopping the operation of the implanted medical device may be performed before connecting, at 2202.

Moreover, the method 2200 may include: electrically disconnecting the implanted medical electrically connected to a controller for operating the medical device. Electrically disconnecting the implanted medical device may be performed after stopping, at 2206, the operation of the implanted medical device.

Electrically disconnecting the implanted medical device may include electrically disconnecting an electrical connector (e.g., the electrical connector portion 102 of the bifunctional connector 100) of the medical from a controller configured to electrically operate the implanted medical device through the electrical connector.

Electrically disconnecting the implanted medical device may include electrically disconnecting an electrical connector (e.g., the electrical connector portion 102 of the bifunctional connector 100) provided to a power wire (e.g., the power wire 116, which may or may not include the tensile wire 106) of the implanted medical device from a controller configured to operate the implanted medical device through the electrical connector.

The method 2200 may include: mechanically disconnecting the wire from the implanted medical device. Mechanically disconnecting the wire from the implanted medical device may be performed after externalization of the externalizable and connectable end portion of the medical device from the subject body (e.g., through the first subject intraluminal access 1900).

Mechanically disconnecting the wire from the implanted medical device may include disconnecting at least one of: (i) a ball connector (e.g., the ball connector 400) and a socket connector (e.g., the socket connector 402) of the wire from a corresponding one of a socket connector (e.g., the socket connector 402) and a ball connector (e.g., the ball connector 400) of the medical device; (ii) a catch connector (e.g., the catch connector 702) and a latch connector (e.g., the latch connector 700) of the wire from a corresponding one of a latch connector (e.g., the latch connector 700) and a catch connector (e.g., the catch connector 702) of the medical device; (iii) a hook connector (e.g., the hook connector 902) and a loop connector (e.g., the loop connector 900) of the wire from a corresponding one of a loop connector (e.g., the loop connector 900) and a hook connector (e.g., the hook connector 902) of the medical device; (iv) a wire (e.g., the wire 1104 of the catheter 1102 of the connection catheter 1100) from a loop connector (e.g., the loop connector 900) of the medical device; and (v) a threaded connector of the wire from a correspondent threaded connector of the medical device (e.g., the threaded connector 1200 and the correspondent threaded connector 1202).

According to the methods 2100 and 2200, the medical device and implanted medical device may be an electrically operable medical device, such as an implantable blood pump. Such medical device may or may not be transcatheterly and/or percutaneously implantable. Also, the medical device may be implantable or implanted in a lumen of a subject body conduit, such as in the vasculature system including the aorta.

As intended herein, an “implantable medical device 114” may include any medical device that is receivable, either partially or totally, inside a natural lumen and/or a surgically made lumen of a subject body conduit, without limitation as to the duration of its presence inside the lumen. For example, such lumen may include a lumen of a subject vasculature, including the subject heart and chambers thereof, as well as any other lumens of any other subject anatomical structures, such as a lumen of a subject lymphatic system. The subject may be a mammalian subject. Also, the “implantable medical device” may include any transcatheterly and/or percutaneously implantable medical device, regardless of whether it is electrically or electronically powered or operated, or not. For example, the “implantable medical device” may be a transcatheterly and/or percutaneously implantable blood pump that is electrically powered or electrically operated.

As intended herein, a “power wire 116” includes a power cable.

As intended herein, a “medical wire 112” includes a wire, a guide wire, cable, and like elongated structure.

1. A connector assembly for an implantable medical device, the connector assembly comprising:
an electrical connector configured to electrically connect the implantable medical device to a controller for operating the implantable medical device through the electrical connector by the controller;
a power cable coupled to the electrical connector such that the implantable medical device is operable through the power cable by the controller, the power cable disposed on a first end portion of the electrical connector; and
a wire configured to be (i) disposed on a second end portion of the electrical connector opposite the first end portion, (ii) advanced inside a lumen of a subject body conduit, (iii) captured inside the lumen of the subject body conduit; and (iv) manipulated to move the implantable medical device inside the lumen of the subject body conduit.

2. The connector assembly according to claim 1, wherein the wire is configured to project from the electrical connector.

3. The connector assembly according to claim 1, wherein the electrical connector is an electrical connector portion, further comprising a mechanical connector portion associated with the electrical connector portion, wherein the wire comprises a mechanical connector configured to removably connect the mechanical connector portion.

4. The connector assembly according to claim 3, wherein the electrical connector portion is configured to be electrically connected to the controller independently of a mechanical connection between the mechanical connector portion and the mechanical connector.

5. The connector assembly according to claim 4, wherein the mechanical connector portion is configured to be mechanically connected to the mechanical connector independently of an electrical connection between the electrical connector portion and the controller.

6. The connector assembly according to claim 3, wherein the electrical connector portion is configured to be electrically connected to the controller when the mechanical connector portion is mechanically unconnected from the mechanical connector.

7. The connector assembly according to claim 6, wherein the mechanical connector portion is configured to be mechanically connected to the mechanical connector only when the electrical connector portion is electrically unconnected from the controller.

8. The connector assembly according to claim 3, wherein the mechanical connector portion is associated with a controller-connecting end portion of the electrical connector portion.

9. The connector assembly according to claim 3, wherein the mechanical connector portion is configured to sustain a pulling force applied thereto that is sufficient to move the implantable medical device inside the lumen of the subject body conduit without disconnection of the mechanical connector portion from the mechanical connector.

10. The connector assembly according to claim 3, wherein the mechanical connector portion comprises one of a ball connector and a socket connector, the wire comprises the other one of the ball connector and the socket connector, the ball connector configured to removably connect to the socket connector.

11. The connector assembly according to claim 3, wherein the mechanical connector portion comprises one of a latch connector and a catch connector, the wire comprises the other one of the latch connector and the catch connector, the latch connector configured to removably connect to the catch connector.

12. The connector assembly according to claim 3, wherein the mechanical connector portion comprises one of a loop connector and a hook connector, the wire comprises the other one of the loop connector and the hook connector, the loop connector configured to removably connect to the hook connector.

13. The connector assembly according to claim 3, wherein the mechanical connector portion comprises one of a first threaded connector and a second threaded connector, the wire comprises the other one of the first threaded connector and the second threaded connector, the first threaded connector configured to removably connect to the second threaded connector.

14. The connector assembly according to claim 1, wherein the wire is configured to be separated in two portions thereof.

15. The connector assembly according to claim 14, wherein the wire is configured to be cut with a cutting pliers.

16. The connector assembly according to claim 1, wherein the wire comprises a free end portion configured to be captured by a snare inside the lumen of the subject body conduit.

17. The connector assembly according to claim 16, wherein the free end portion is more flexible than a remainder portion of the wire.

18. The connector assembly according to claim 1, wherein the wire is a guidewire.

19. A method of using a connector assembly of an implantable medical device, wherein the connector assembly comprises an electrical connector and a wire disposed on a controller-connecting end portion of the electrical connector and capturable inside a lumen of a subject body conduit by an operator, the method comprising:
manipulating the wire; and
electrically connecting the electrical connector to a controller for operating the implantable medical device through the electrical connector by the controller.

20. The method according to claim 19, wherein the electrical connector is an electrical connector portion, the connector assembly further comprising a mechanical connector portion associated with the electrical connector portion, said manipulating the wire comprises disconnecting the wire from the mechanical connector portion.

21. The method according to claim 20, wherein said disconnecting the wire from the mechanical connector portion comprises disconnecting the wire from the mechanical connector portion independently of disconnecting an electrical connection between the electrical connector portion and the controller.

22. The method according to claim 19, wherein said manipulating the wire comprises separating the wire in two portions thereof.

23. The method according to claim 22, wherein said separating the wire in two portions thereof comprises cutting the wire in two portions thereof with a cutting pliers by the operator.

24. The method according to claim 19, wherein said manipulating the wire comprises folding the wire to electrically connect the electrical connector to the controller.

25. The method according to claim 19, wherein said manipulating the wire comprises passing the wire through an electrical connector of the controller to electrically connect the electrical connector to the controller.

26. The method according to claim 19, wherein the connector assembly further comprises a power cable disposed on an end portion of the electrical connector opposite the controller-connecting end portion, the power cable coupled to the electrical connector such that the implantable medical device is operable through the power cable by the controller.

27. The method according to claim 19, wherein the wire projects from the electrical connector.

28. The method according to claim 19, wherein the wire is a guide wire.
